(19) United States
(12) Patent Application Publication
(10) Pub. No.: US 2011/0104074 A1
(43) Pub. Date: May 5, 2011

(54) METHODS FOR TARGETED CANCER TREATMENT AND DETECTION

(75) Inventor: Sham S. Kakar, Prospect, KY (US)

(73) Assignee: UNIVERSITY OF LOUISVILLE RESEARCH FOUNDATION, INC., Louisville, KY (US)

(21) Appl. No.: 12/999,404

(22) PCT Filed: Jun. 18, 2009
(86) PCT No.: PCT/US09/47816

§ 371 Date: Dec. 16, 2010

Related U.S. Application Data
(60) Provisional application No. 61/073,635, filed on Jun. 18, 2008.

(51) Int. Cl. A61K 49/18; A61K 38/22; A61K 9/127; A61K 49/00

(52) U.S. Cl. 424/9.32; 514/9.7; 424/450; 424/9.1

(57) ABSTRACT

A method for treating a cancer is provided that comprises identifying a subject in need of treatment for a cancer and administering to the subject a composition that is comprised of a plurality of a ligand for a luteinizing hormone-releasing hormone receptor and a plurality of a chemotherapeutic agent, where each ligand and each chemotherapeutic agent are conjugated to a nanoparticle. Further provided are methods for detecting a cancer in a subject that comprise administering to a subject an imaging agent comprised of a plurality of a ligand for a luteinizing hormone-releasing hormone receptor conjugated to a nanoparticle.

### RELATED APPLICATIONS

This application claims priority from U.S. Provisional Application Ser. No. 61/073,635, filed Jun. 18, 2008, the entire disclosure of which is incorporated herein by this reference.

### GOVERNMENT INTEREST

Subject matter described herein was made with U.S. Government support under Grant Number W81XWH-06 awarded by the Department of Defense. The government has certain rights in the described subject matter.

### TECHNICAL FIELD

The presently-disclosed subject matter relates to methods for targeted cancer treatment and detection. In particular, the presently-disclosed subject matter relates to methods for targeted cancer treatment that make use of a nanoparticle conjugated with a plurality of ligands for a luteinizing hormone-releasing hormone receptor and a plurality of a chemotherapeutic agent, and methods for targeted cancer detection that make use of a nanoparticle conjugated with a plurality of ligands for a luteinizing hormone-releasing hormone receptor.

### BACKGROUND

Despite the progress that has been made in the treatment and diagnosis of various cancers, cancer continues to be a significant health problem in the United States and other developed countries with one in four deaths currently being attributed to cancer. For example, ovarian cancer is the leading cause of gynecological malignancy and, in 2008, approximately 21,650 new cases of ovarian cancer and 15,520 deaths were expected in the United States alone (8). Indeed, the cure rate for ovarian cancer diagnosed at an advanced stage is less than 20% due the absence of symptoms in the early stage of the disease. Unlike colon and cervical cancers, ovarian cancer has no identifiable precancerous lesions that can be used for screening. In fact, screening by transvaginal ultrasonography and radioimmunoassay for CA-125 show low specificity and sensitivity, which make these two methods unreliable for detecting the initial stages of disease.

Currently, the primary treatment modality for ovarian cancer is cytoreductive surgery followed by adjuvant chemotherapy, radiotherapy, or both (9,10). While this strategy has been successfully employed in a number of patients, it is always accompanied by cytotoxicity to normal cells and tissues, and multidrug resistance (MDR). For ovarian cancer treatment, a number of drugs including cisplatin [cis-dichlorodiamine platinum (CDDP)], carboplatin, oxaliplatin, paclitaxel, topotecan, and DOXIL® are clinically used. These drugs are typically effective in suppressing ovarian tumors; however, due to the high doses of each drug that are administered to a patient, and which are required to achieve effective tumor cell death, almost every drug presently used is accompanied by cytotoxicity to normal cells and tissues.

In any event, known methods of detecting and treating many cancers are less than sufficient. Due to the low specificity and sensitivity of detection methods, a particular cancer cannot be reliably diagnosed or can only be diagnosed after it has reached an advanced stage. Further, none of the known treatment methods for certain cancers sufficiently address the administration of a more potent and more specific drug for the treatment of a cancer, which exhibits minimal or no cytotoxicity to normal cells or tissues.

### SUMMARY

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

In some embodiments of the presently-disclosed subject matter, a method for treating a cancer in a subject is provided. In some embodiments, the method comprises identifying a subject in need of a treatment for a cancer and administering to the subject an effective amount of a composition that is comprised of a plurality of a chemotherapeutic agent and a plurality of a ligand for a luteinizing hormone-releasing hormone (LHRH) receptor, where both the ligand for the LHRH receptor and the chemotherapeutic agent are conjugated to a nanoparticle. In some embodiments, the composition is PEGylated.

In some embodiments of the presently-disclosed methods, the ligand for an LHRH receptor comprises LHRH or an analog thereof. In some embodiments, the ligand can be [D-Trp⁶] LHRH. In some embodiments, the plurality of the ligand for an LHRH receptor comprises about 150 to about 250 molecules of the ligand. In some embodiments, about 200 molecules of the ligand for an LHRH receptor can be conjugated to a nanoparticle.

In some embodiments of the presently-disclosed therapeutic methods, the plurality of chemotherapeutic agents, which are conjugated to the nanoparticle, comprises about 50 to about 100 molecules of the chemotherapeutic agent. In some embodiments, the plurality of the chemotherapeutic agent comprises about 75 molecules of the chemotherapeutic agent. In some embodiments, the chemotherapeutic agent is cisplatin, carboplatin, paclitaxel, topotecan, or doxorubicin. In some embodiments, the concentration of the chemotherapeutic agent that is delivered to a subject via the presently-disclosed therapeutic methods, is about 5 μg/kg to about 15 μg/kg. In some embodiments, the concentration of the chemotherapeutic agent is about 9.6 μg/kg.

In some embodiments, the nanoparticle, which is conjugated to the plurality of ligands for an LHRH receptor and the plurality of chemotherapeutic agents, is a gold nanoparticle or a liposome. In some embodiments, the nanoparticle is a pure gold nanoparticle. Further, in some embodiments, the nanoparticle is a pure gold nanoparticle, which is conjugated to about 200 molecules of a [D-Trp⁶] luteinizing hormone-releasing hormone and to about 75 molecules of doxorubicin.

Further provided, in some embodiments of the presently-disclosed subject matter, are methods for detecting a cancer in a subject. In some embodiments, a method for detecting a cancer is provided that comprises administering to a subject an effective amount of an imaging agent that comprises a plurality of a ligand for an LHRH receptor conjugated to a nanoparticle such that the ligand targets the imaging agent to the cancer in the subject. In some embodiments, the agent targeted to the cancer is then imaged to thereby detect the cancer in the subject. In some embodiments, the agent is imaged by magnetic resonance imaging.

With regard to the various cancers that can be treated or detected by the presently-disclosed methods, in some embodiments, the cancer expresses an LHRH receptor. In some embodiments, the cancer is selected from breast cancer, ovarian cancer, prostate cancer, liver cancer, endometrial cancer, pancreatic cancer, melanoma, testicular cancer, pituitary cancer, uterine cancer, Non-Hodgkin's lymphoma, and renal cell carcinoma.

Advantages of the presently-disclosed subject matter will become evident to those of ordinary skill in the art after a study of the description, Figures, and non-limiting Examples in this document

### BRIEF DESCRIPTION OF THE DRAWINGS

FIG. 1 is a schematic representation of an exemplary conjugation reaction between gold nanoparticles (NGP), [D-Trp⁶] luteinizing hormone-releasing hormone (LHRH), and doxorubicin (DOX) in accordance with the presently-disclosed subject matter, and showing a plurality of LHRH and DOX molecules conjugated to a single NGP.

FIG. 2 is a graph showing the results of a luciferase assay and depicting the binding of [D-Trp⁶] LHRH (LHRH) and gold nanoparticles conjugated with [D-Trp⁶] LHRH and doxorubicin (LHRH-NP-DOX) to an LHRH receptor, where the relative light units (RLU; y-axis) are plotted against the concentrations (nM) of LHRH and LHRH-NP-DOX that were utilized in the experiments (x-axis).

FIGS. 3A-3C include graphs depicting the effects of various concentrations of doxorubicin (Dox), LHRH, Dox-LHRH conjugates, Dox-gold nanoparticle conjugates (Dox-NP), and gold nanoparticles conjugated with [D-Trp⁶] LHRH and doxorubicin (Dox-NP-LHRH) on the cell viability of A2780 ovarian tumor cells. FIG. 3A shows the effects of the various agents on A2780 cells at 24 hrs, while FIGS. 3B and 3C depict the effects of the various agents on the cells at 48 and 72 hrs, respectively.

FIGS. 4A-4C include graphs depicting the effects of various concentrations of doxorubicin (Dox), LHRH, LHRH-gold nanoparticle conjugates (LHRH-NP), Dox-gold nanoparticle conjugates (Dox-NP), and gold nanoparticles conjugated with [D-Trp⁶] LHRH and Dox (Dox-NP-LHRH) on the cell viability of CAOV3 ovarian tumor cells. FIG. 4A shows the effects of the various agents on CAOV3 cells at 24 hrs, while FIGS. 4B and 4C depict the effects of the various agents on the cells at 48 and 72 hrs, respectively.

FIGS. 5A-5C include graphs depicting the effects of various concentrations of doxorubicin (Dox), LHRH, LHRH-gold nanoparticle conjugates (LHRH-NP), Dox-gold nanoparticle conjugates (Dox-NP), and gold nanoparticles conjugated with [D-Trp⁶] LHRH and Dox (Dox-NP-LHRH) on the cell viability of UCI ovarian tumor cells. FIG. 5A shows the effects of the various agents on UCI cells at 24 hrs, while FIGS. 5B and 5C depict the effects of the various agents on the cells at 48 and 72 hrs, respectively.

FIG. 6 is a graph depicting the uptake of doxorubicin (Dox) by A2780 ovarian tumor cells, where the cells were incubated with free Dox (Dox), Dox conjugated to gold nanoparticles (Dox-NP), or Dox conjugated to gold nanoparticles that are also conjugated with LHRH (LHRH-NP-DOX).

FIG. 7 includes images showing the uptake of fluorescence-labeled particles by A2780 ovarian tumor cells, and including images of A2780 cells that were incubated with PBS (Control), fluorescent nanoparticles (FNP), doxorubicin conjugated fluorescent nanoparticles (Dox-FNP), or LHRH and DOX conjugated fluorescent nanoparticles (LHRH-FNP-DOX).

FIG. 8 is a graph depicting the effect of LHRH-NP-DOX conjugated particles on the suppression of tumor growth in nude mice subcutaneously injected with A2780 cells, where tumor volume (y-axis) is plotted against the various experimental groups.

FIG. 9 is a graph depicting the uptake of doxorubicin by various tissues in animals that were previously injected with A2780 ovarian tumor cells, where the animals subsequently received injections of free Dox, Dox conjugated to gold nanoparticles (Dox-NP), or Dox conjugated to gold nanoparticles that are also conjugated with LHRH (LHRH-NP-Dox).

FIGS. 10A-10B include images showing the uptake of fluorescent nanoparticles conjugated with Dox (FIG. 10A) and the uptake of fluorescent nanoparticles conjugated with LHRH and Dox (FIG. 10B) by various sub-cellular organelles in A2780 cells.

### BRIEF DESCRIPTION OF THE SEQUENCE LISTING

SEQ ID NO: 1 is an amino acid sequence of a luteinizing hormone-releasing hormone (LHRH).

SEQ ID NO: 2 is an amino acid sequence of a [D-Trp⁶] luteinizing hormone-releasing hormone analog.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The luteinizing hormone-releasing hormone (LHRH) receptor is a seven transmembrane receptor and belongs to the family of G-protein-coupled receptors. Binding of a ligand, such as the endogenous LHRH receptor ligand, LHRH, to the receptor induces confirmation changes, and induces desensitization and internalization of the ligand-LHRH receptor as a complex (19). Once the receptor is internalized, it is translocated to the endosomes and sorted out to the lysosomes. In lysosomes, due to a low pH, the ligand is detached and most of the receptor protein undergoes degradation, while a small amount of the receptor is recycled to the membrane (19). The LHRH receptor possesses a unique structure and is the smallest member of the G-protein coupled receptor family (20,21). It lacks a C-terminal domain required for the desensitization and internalization of the receptor (20,21). As a result, the LHRH receptor undergoes desensitization and internalization at a much slower rate as compared to other G-protein coupled receptors.

As noted above, LHRH, which is also known as gonadotropin-releasing hormone (GnRH), is an endogenous ligand for the LHRH receptor. LHRH is a hypothalamic decapeptide with a sequence of pGlu-His-Trp-Ser-Tyr-Gly-Leu-Arg-Pro-Gly-NH₂ (SEQ ID NO: 1). LHRH is synthesized by hypothalamic neurons and is released into the portal blood, where it then travels to and stimulates the synthesis and secretion of gonadotropins (i.e., follicle stimulating hormone (FSH) and luteinizing hormone (LH)) in the anterior pituitary. LHRH and its receptor have also been reported as being expressed in a variety of cancerous tumors (19) including, but not limited to, those tumors that are found in breast cancer, ovarian cancer, prostate cancer, liver cancer, endometrial cancer, pancreatic cancer, melanoma, testicular cancer, pituitary cancer, uterine cancer, Non-Hodgkin's lymphoma, and renal cell carcinoma. For example, approximately 80% of human ovarian and endometrial cancers and approximately 50% of breast cancers express LHRH and its receptors as part of an autocrine regulatory system. However, LHRH and its receptor are not typically expressed at a detectable level in most visceral tissues and organs.

The present invention is based on the above-described differential expression of LHRH and its receptors in a number of cancers, including how this differential expression can be advantageously used to treat a cancer in a subject. Briefly, LHRH can be used to specifically direct chemotherapeutic agents to cancer cells to inhibit tumor cell growth and metastasis without adversely affecting the surrounding non-cancerous cells and tissues. It has now been surprisingly determined that by conjugating a plurality of LHRH receptor ligands and a plurality of a particular chemotherapeutic agent to a nanoparticle, cancer cells can be specifically targeted and a higher concentration of the particular chemotherapeutic agent can be selectively delivered to the cancer cells in order to induce cell death. That is, the nanoparticles can be conjugated to a number of LHRH molecules, or analogs thereof, and a number of molecules of a particular chemotherapeutic agent such that the chemotherapeutic agent is specifically directed to only the cancer cells and toxicity to normal cells and tissues is thereby avoided. In this manner, specificity and potency of the of the chemotherapeutic agent is beneficially enhanced.

In some embodiments of the presently-disclosed subject matter, a method for treating a cancer is provided. As used herein, the terms “treatment” or “treating” relate to any treatment of a cancer, including, but not limited to, prophylactic treatment and therapeutic treatment As such, the terms treatment or treating include, but are not limited to: inhibiting the progression of a cancer; inhibiting the spread or metastasis of a cancer; arresting or preventing the development of a cancer; reducing the severity of a cancer; ameliorating or relieving symptoms associated with a cancer; and causing a regression of the cancer or one or more of the symptoms associated with the cancer.

In some embodiments, a method for treating a cancer is provided that comprises identifying a subject in need of treatment for a cancer and administering to the subject an effective amount of a composition that comprises a plurality of a chemotherapeutic agent and a plurality of a ligand for an LHRH receptor, where each chemotherapeutic agent and each ligand are conjugated to a nanoparticle. In some embodiments, the ligand for a luteinizing hormone-releasing hormone receptor comprises a luteinizing hormone-releasing hormone, such as that provided in SEQ ID NO: 1, or an analog thereof

The term “ligand” as used herein in reference to ligands for a LHRH receptor is meant to refer to any substance that is capable of forming a complex with an LHRH receptor such that conformational changes are induced and the ligand-LHRH receptor is internalized as a complex to the interior of a particular cell, such as a cancer cell. Examples of ligands for an LHRH receptor that can be used in accordance with the presently-disclosed subject matter include, but are not limited to, LHRH molecules and analogs thereof

The term “analog” as used herein in reference to LHRH analogs is meant to refer to synthetic or natural peptides that resemble LHRH in structure and function, and which can be used to specifically direct a conjugated nanoparticle to a cancer cell. Various LHRH analogs are known to those or ordinary skill in the art and include, but are not limited to, gonadorelin, leuprolide, leuprorelin, lecirelin, histerlin, buserdin, and triptorelin. Typically, an LHRH analog is comprised of a peptide sequence that is similar to an endogenous LHRH peptide sequence, but in which one or more residues have been replaced, deleted, or otherwise modified with a desired chemical moiety. For example, triptorelin, which is used herein interchangeably with the term [D-Trp⁶] LHRH, is an LHRH analog in which a D-steroisomer of tryptophan replaces a glycine residue as the sixth residue in the LHRH peptide chain, and in which the penultimate proline residue has been removed. In some embodiments of the presently-disclosed subject matter, the ligand for a LHRH receptor comprises a [D-Trp⁶] LHRH.

The term “cancer” refers to all types of cancer or neoplasm or malignant tumors found in animals, including leukemias, carcinomas, melanoma, and sarcomas. Examples of cancers are cancer of the brain, bladder, breast, cervix, colon, head and neck, kidney, lung, non-small cell lung, mesothelioma, ovary, prostate, sarcoma, stomach, uterus and Medulloblastoma. In some embodiments of the presently-disclosed subject matter, the cancer is a cancer that expresses a luteinizing hormone-releasing hormone receptor. In some embodiments, identifying a subject in need of treatment for a cancer includes identifying the subject as having a cancer that expresses an LHRH receptor. For example, to identify a subject as having a cancer that expresses an LHRH receptor, a biopsy or sample of the cancer can be taken and the presence of an LHRH receptor within the cancer can be determined by a variety of methods known to those or ordinary skill in the art to thereby determine whether the subject has a cancer that expresses an LHRH receptor.

As noted, LHRH receptors are typically expressed at non-detectable levels in the majority of the visceral organs of a subject, but have been found to be expressed in a number of cancers. In some embodiments of the presently-disclosed subject matter, this differential expression can be used to specifically target a nanoparticle, conjugated with a plurality of a ligand for an LHRH receptor and a plurality of a chemotherapeutic agent, to a cancer cell in a subject. In some embodiments, the cancer that expresses the LHRH receptor is selected from breast cancer, ovarian cancer, prostate cancer, liver cancer, endometrial cancer, pancreatic cancer, melanoma, testicular cancer, pituitary cancer, uterine cancer, Non-Hodgkin's lymphoma, and renal cell carcinoma.

As used herein, the term “chemotherapeutic agent” refers to an agent that is capable of “treating” a cancer, as defined herein. For example, the chemotherapeutic agent can kill cancer cells, prevent or inhibit the development of cancer cells, induce apoptosis in cancer cells, reduce the growth rate of cancer cells, reduce the incidence or number of metastases, reduce tumor size, inhibit tumor growth, reduce the blood supply to a tumor or cancer cells, promote an immune response against cancer cells or a tumor, prevent or inhibit the progression of cancer, or increase the lifespan of a subject with cancer.

Examples of chemotherapeutic agents include, but are not limited to, platinum coordination compounds such as cisplatin, carboplatin or oxaliplatin; taxane compounds, such as paclitaxel or docetaxel; topoisomerase I inhibitors such as camptothecin compounds for example irinotecan or topotecan; topoisomerase II inhibitors such as anti-tumor podophyllotoxin derivatives for example etoposide or teniposide; anti-tumor vinca alkaloids for example vinblastine, vincristine or vinorelbine; anti-tumor nucleoside derivatives for example 5-fluorouracil, gemcitabine or capecitabine; alkylating agents, such as nitrogen mustard or nitrosourea for example cyclophosphamide, chlorambucil, carmustine or lomustine; anti-tumor anthracycline derivatives for example daunorubicin, doxorubicin, idarubicin or mitoxantrone; HER2 antibodies for example trastuzumab; estrogen receptor antagonists or selective estrogen receptor modulators for example tamoxifen, toremifene, droloxifene, faslodex or raloxifene; aromatase inhibitors, such as exemestane, anastrozole, letrazole and vorozole; differentiating agents such as retinoids, vitamin D and retinoic acid metabolism blocking agents (RAMBA) for example accutane; DNA methyl transferase inhibitors for example azacytidine; kinase inhibitors for example flavoperidol, imatinib mesylate or gefitinib; farnesyltransferase inhibitors; HDAC inhibitors; other inhibitors of the ubiquitin-proteasome pathway for example VELCADE®; or YONDELIS®. In some embodiments of the presently-disclosed subject matter, the chemotherapeutic agent is selected from cisplatin, oxaliplatin, paclitaxel, topotecan, and doxorubicin. In some embodiments, the chemotherapeutic agent comprises doxorubicin.

The term “nanoparticle” is used herein to refer to nano-sized particles that are approximately 200 nm or less in size and exhibit high rates of diffusion due to a large surface area to volume ratio of the individual nanoparticles. A variety of nanoparticles are known to those of ordinary skill in the art and are typically referred to based on their size, shape, the method by which they are manufactured, and their particular composition. For example, certain terms for nanoparticles relate to the shape of the individual nanoparticles, and include such terms such as nanospheres, nanocups, and nanorods.

Regardless of the particular terms that are used to refer to a nanoparticle, however, nanoparticles can be broadly classified into two groups, organic and inorganic. Inorganic nanoparticles are typically comprised of a metal (e.g., gold, iron, titanium, silver, copper, platinum, or cobalt), a combination of metals, or at least a metal core that defines the fluorescence, magnetic, optical, and electronic properties of the nanoparticle. Further, inorganic nanoparticles can also include an outer organic layer that protects the core from degradation in a variety of physiological environments and also acts as a conjugating medium that is capable of forming various bonds with a variety of other particles. Organic nanoparticles, on the other hand, are comprised of carbon-based structures and include, but are not limited to, nanoparticles such as dendrimers, liposomes, emulsions, carbon nanotubes, various polymers, sol gel, transparent materials, colloids, and pH-sensitive nanoparticles. For additional guidance regarding the various types of nanoparticles and their respective properties, see, e.g., Pohlgeers, et al., “Implication of nanoparticles in diagnosis and treatment of cancer: the future of oncology” Cell Death Signaling and Cancer. 2009: 145-197, which is incorporated herein by this reference.

In some embodiments of the presently-disclosed subject matter, the nanoparticle is a gold nanoparticle or a liposome. The term “liposome” is used herein to refer to vesicles comprised of one or more bilayer membranes. The membranes can be formed from materials such as naturally-derived phospholipids with mixed lipid chains, or of pure surfactant components like dioleoylphosphatidylethanolamine (DOPE). Liposomes typically, but not by definition, contain a core of aqueous material that can be used to carry a therapeutic agent. The delivery of therapeutic agents using liposome carriers is well known in the art and a variety of methods are available for preparing liposomes, such as those as described in, e.g., Szoka et al. Ann. Rev. Biophys. Bioeng. 9:467 (1980);and U.S. Pat. Nos. 4,394,448; 4,235,871; 4,501,728; 4,837,028; and 5,019,369, which are each incorporated herein by this reference. Of course, the selection of a particular lipid that is used to form a liposome can vary and is typically guided by the desired size, acid lability, and stability of the liposomes for a particular application.

As noted, in some embodiments of the presently-disclosed subject matter, the nanoparticle can be a gold nanoparticle, such as a pure gold nanoparticle. Gold nanoparticles are chemically-inert and non-toxic in biological and physiological systems. Additionally, gold nanoparticles display an optical absorption in the near-infrared spectrum, thereby making them suitable for use as a contrast agent for imaging applications. To conjugate LHRH receptor ligands, such as LHRH peptides and analogs thereof, as well as chemotherapeutic agents to gold nanoparticles, a variety of methods known to those of ordinary skill in the art can be used. For example, gold nanoparticles can first be coated with an appropriate surfactant layer, such as a tannin layer, to provide a biocompatible surface onto which various molecules can be attached. LHRH molecules, or analogs thereof, can then be conjugated to the surface of the gold nanoparticles via their N-terminal amine group, and chemotherapeutic agents can be conjugated to the surface of the gold nanoparticles via various reactive groups that are present in the chemical structures of the individual chemotherapeutic agents, such as the thiol group of doxorubicin.

The term “conjugate,” or grammatical variations thereof, is used herein to refer to the association of one or more molecules (e.g., a nanoparticle and a chemotherapeutic agent) to form a composition as disclosed herein. For example, conjugation can occur through the joining together of one or more chemical moieties, either covalently or non-covalently, or can occur via the encapsulation of one or more molecules inside another. As another example of conjugation, the joining together of one or more chemical moieties can occur via the encapsulation of one or more molecules inside another in conjunction with the attachment of one or molecules to each other, such as is the case with liposomes where a chemotherapeutic agent can be encapsulated by the bilayer membrane and one or more targeting molecules, such as an LHRH molecule, can be attached to the bilayer membrane itself to provide targeting of the liposome to a cancer cell.

In some embodiments, the plurality of the ligand for an LHRH receptor that is conjugated to a nanoparticle can comprise about 150, about 155, about 160, about 165, about 170, about 175, about 180, about 185, about 190, about 195, about 200, about 205, about 210, about 215, about 220, about 225, about 230, about 235, about 240, about 245, or about 250 molecules of the ligand. In some embodiments, the plurality of the ligand for a LHRH receptor comprises about 150 to about 250 molecules of the ligand. In some embodiments, the plurality of the ligand for a LHRH receptor comprises about 200 molecules of the ligand.

In some embodiments, the plurality of the chemotherapeutic agent that is conjugated to a nanoparticle can comprise about 50, about 55, about 60, about 65, about 70, about 75, about 80, about 85, about 90, about 95, or about 100 molecules of the chemotherapeutic agent. In some embodiments, the plurality of the chemotherapeutic agent comprises about 50 to about 100 molecules of the chemotherapeutic agent. In some embodiments, the plurality of the chemotherapeutic agent comprises about 75 molecules of the chemotherapeutic agent.

In some embodiments of the presently-disclosed subject matter, a method of treating a cancer is provided that includes administering a composition that is comprised of pure gold nanoparticle conjugated to about 200 molecules of a [D-Trp⁶] LHRH as a ligand for an LHRH receptor and conjugated to about 75 molecules of doxorubicin as a chemotherapeutic agent.

In some embodiments of the presently-disclosed subject matter, the compositions described herein can further be modified by the attachment of one or more pH-sensitive components including, but not limited to: unsaturated dioleoylphosphatidylethanolamine (DOPE); pH-sensitive nanogels; pH-sensitive polymers such as polyacrylic acid and chitosan; and pH-sensitive hydrogels. For example, DOPE can be combined with an LHRH-targeted liposome formulation that includes a chemotherapeutic agent such that the liposome fuses to an endovacuolar membrane in a cell after being endocytosed. The endovacuolar membrane has a lower pH that destabilizes the liposome membrane, and thereby provides for the release of the particular chemotherapeutic agent into the cytoplasm of the cell.

Similarly, in some embodiments, the compositions described herein can comprise degradable nanoparticles, such as degradable pH-sensitive liposomes incorporating polyphosphazene polymers, that will undergo degradation in lysosomes to thereby release a chemotherapeutic agent into a cell. For example, pH-sensitive polymers that swell in a basic pH environment and shrink or undergo degradation in an acid environment can be incorporated into a liposome, which is conjugated to a plurality of LHRH molecules and chemotherapeutic agents, such that when the liposome is internalized into a cancer cell, the chemotherapeutic agent is released into the endosomes or lysosomes of the cell.

In some embodiments of the presently-disclosed subject matter, additional components can be added to the compositions to increase the half-life and decrease the immunogenicity of the compositions in a subject. For example, in some embodiments, the compositions are PEGylated by adding a polyethylene glycol (PEG) coating to the compositions, such as what is described in U.S. Pat. Nos. 5,013,556 and 5,213,804, which are incorporated herein by this reference. In some embodiments, gold nanoparticles are PEGylated. In other embodiments, a gold nanoparticle is first conjugated to a plurality of a chemotherapeutic agent and a plurality of a LHRH receptor ligand, and then the entire composition is PEGylated.

For administration of a composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich, et al., (1966) Cancer Chemother. Rep. 50: 219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Cancer Chemother. Rep. 1996; 50: 219-244). Briefly, to express a mg/kg dose in any given species as the equivalent mg/sq m dose, multiply the dose by the appropriate kg factor. In an adult human, 100 mg/kg is equivalent to 100 mg/kg×37 kg/sq m=3700 mg/m².

Suitable methods for administering a composition in accordance with the methods of the present invention include, but are not limited to, systemic administration, parenteral administration (including intravascular, intramuscular, intra-arterial administration), subcutaneous administration, local injection, and intra-peritoneal administration. Where applicable, continuous infusion can enhance drug accumulation at a target site (see, e.g., U.S. Pat. No. 6,180,082). Of course, the particular mode of administration used in accordance with the methods of the present invention depends on various factors, including but not limited to the vector and/or drug carrier employed, the severity of the condition to be treated, and mechanisms for metabolism or removal of the composition following administration.

Injectable formulations of the compositions used herein can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). For intravenous injections, water-soluble versions of the compounds can be administered by the drip method, whereby a composition and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically-acceptable oil base, such as an ester of a long chain fatty acid, (e.g., ethyl oleate).

The term “effective amount” is used herein to refer to an amount of the composition (e.g., a composition comprising a nanoparticle conjugated to a plurality of a ligand for an LHRH receptor and a plurality of a chemotherapeutic agent) sufficient to produce a measurable biological response (e.g., inhibition of the development of tumor cells or a reduction in the number of tumor cells). Actual dosage levels of active ingredients in a therapeutic composition of the presently-disclosed subject matter can be varied so as to administer an amount of the active compound(s) that is effective to achieve the desired therapeutic response for a particular subject and/or application. The selected dosage level will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art of medicine.

For additional guidance regarding formulation and dose, see U.S. Pat. Nos. 5,326,902 and 5,234,933; PCT International Publication No. WO 93/25521; Berkow, et al., (1997) The Merck Manual of Medical Information, Home ed. Merck Research Laboratories, Whitehouse Station, N.J.; Goodman, et al., (2006) Goodman & Gilman's the Pharmacological Basis of Therapeutics, 11th ed. McGraw-Hill Health Professions Division, New York; Ebadi. (1998) CRC Desk Reference of Clinical Pharmacology. CRC Press, Boca Raton, Fla.; Katzung, (2007) Basic & Clinical Pharmacology, 10th ed. Lange Medical Books/McGraw-Hill Medical Pub. Division, New York; Remington, et al., (1990) Remington's Pharmaceutical Sciences, 18th ed. Mack Pub. Co., Easton, Pa.; Speight, et al., (1997) Avery's Drug Treatment: A Guide to the Properties, Choice, Therapeutic Use and Economic Value of Drugs in Disease Management, 4th ed. Adis International, Auckland/Pa.; and Duch, et al., (1998) Toxicol. Lett. 100-101:255-263.

In some embodiments of the presently-disclosed subject matter, the dose of the chemotherapeutic agent that is administered to the subject is about 1, about 2, about 3, about 4, about 5, about 6, about 7, about 8, about 9, about 10, about 11, about 12, about 13, about 14, about 15, about 16, about 17, about 18, about 19, or about 20 μg/kg. In some embodiments, the dose of the chemotherapeutic agent is about 5 to about 15 ∥g/kg. In some embodiments, the concentration of the chemotherapeutic agent is about 9.6 μg/kg. By conjugating a plurality of ligands for an LHRH receptor and a plurality of a chemotherapeutic agent to a nanoparticle as disclosed herein, the methods of the presently-disclosed subject matter allow for significantly lower doses of a chemotherapeutic agent to be administered to a subject in need of cancer treatment, but yet still are capable of delivering an amount of a chemotherapeutic agent to a cancer cell that is sufficient to induce cancer cell death. For example, the inventors have surprisingly discovered that by employing the presently-disclosed conjugated nanoparticles in a method for treating a cancer, the dose of doxorubicin that is administered to a subject is approximately 1000-fold less than doses that have been previously described in the literature for the treatment of a cancer in a subject.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

Further provided, in some embodiments of the presently-disclosed subject matter are pharmaceutical compositions that can be comprised of any of the compositions disclosed herein. In some embodiments, a pharmaceutical composition is provided that comprises a nanoparticle, as disclosed herein, that is conjugated to a plurality of a ligand for an LHRH receptor and a plurality of a chemotherapeutic agent. In some embodiments, a pharmaceutical composition is provided that comprises a gold nanoparticle that is conjugated to about 200 molecules of [D-Trp⁶] LHRH and to about 75 molecules of doxorubicin.

Any pharmaceutical composition of the presently-disclosed subject matter can be provided in the form of a pharmaceutically acceptable salt or solvate. A salt can be formed using a suitable acid and/or a suitable base. Suitable acids that are capable of forming salts with the pharmaceutical compositions of the presently disclosed subject matter include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, or the like. Suitable bases capable of forming salts with the pharmaceutical compositions of the presently disclosed subject matter include inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine, and the like), and optionally substituted ethanolamines (e.g. ethanolamine, diethanolamine, and the like).

As used herein, the term “solvate” refers to a complex or aggregate formed by one or more molecules of a solute, e.g., a composition or a pharmaceutically-acceptable salt thereof, and one or more molecules of a solvent. Such solvates are typically crystalline solids having a substantially fixed molar ratio of solute and solvent. Representative solvents include, but are not limited to, water, methanol, ethanol, isopropanol, acetic acid, and the like. When the solvent is water, the solvate formed is a hydrate. As such, the term “pharmaceutically-acceptable salt or solvate thereof” is intended to include all permutations of salts and solvates, such as a solvate of a pharmaceutically-acceptable salt of a composition.

Still further provided, in some embodiments of the presently-disclosed subject matter, are methods for detecting a cancer in a subject. In some embodiments, a method for detecting a cancer is provided that comprises administering to the subject an effective amount of an imaging agent comprising a plurality of a ligand for an LHRH receptor conjugated to a nanoparticle such that the ligand targets the imaging agent to the cancer in the subject. In some embodiments, the agent, which is targeted to the cancer, is then imaged to thereby detect a cancer in the subject.

The terms “detect” and “detecting” are used herein to refer to determining the presence, absence, or the amount of cancer in a subject. As such, “detecting a cancer” can refer to determining whether a cancer is present or absent in a particular subject, as well as quantifying the amount (e.g., tumor size) of a cancer in a subject. For example, to detect a cancer in a subject, gold nanoparticles that are conjugated to a plurality of a ligand for an LHRH receptor can first be administered to a subject such that the gold nanoparticles are specifically directed to a cancer expressing an LHRH receptor. The binding of the conjugated nanoparticles to the LHRH receptors on the cancer cells causes the internalization of the conjugated nanoparticles, which allows the internalized gold nanoparticles to then be imaged to thereby determine whether a cancer is present or absent in a particular subject. As another example, to quantify the amount of a cancer in a subject, the intensity of a signal that is obtained after imaging the conjugated gold nanoparticles can be measured and used to assess the size of a particular tumor as well as the metastatic spread of cancer cells within a particular subject such that the amount of a cancer in a subject can be quantified.

The term “imaging” is used herein to refer to methods by which one of ordinary skill in the art can visualize an imaging agent, such as those described herein, within a particular subject. For example, a variety of methods for imaging a gold nanoparticle are known to those of ordinary skill in the art and include, but are not limited to, methods such as magnetic resonance imaging (MRI), computed tomography (CT), or Raman scattering techniques. In some embodiments, the agent is imaged by magnetic resonance imaging.

The presently-disclosed subject matter is further illustrated by the following specific but non-limiting examples. Some of the following examples are prophetic, notwithstanding the numerical values, results and/or data referred to and contained in the examples. The following examples also may include compilations of data that are representative of data gathered at various times during the course of development and experimentation related to the present invention.

### EXAMPLES

### Example 1

### Cross-Linking of a Luteinizing Hormone-Releasing Hormone Analog and Doxorubicin to Gold Nanoparticles

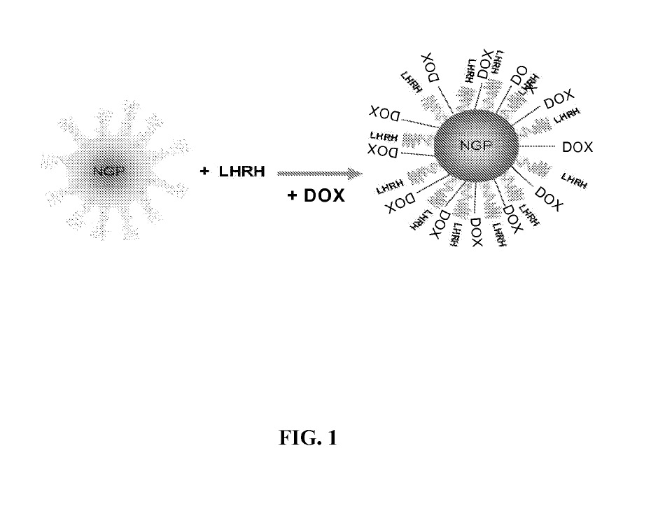

A luteinizing hormone-releasing hormone analog, [D-Trp⁶] LHRH (MW=1311.45) and doxorubicin (DOX) were both conjugated to gold nanoparticles (NPs), which were prepared as described previously (81). [D-Trp⁶] LHRH (Triptorelin; referred to hereinafter as LHRH) has an amino acid sequence of p-Glu-His-Trp-Ser-Tyr-D-Trp-Leu-Arg-Gly-NH₂ (SEQ ID NO: 2) and was used to provide tumor specificity to the NPs (80). LHRH was coupled to the surface of appropriately prepared NPs via its N-terminal amine group. For control purposes, the non-specific peptide, thyrotrophic-releasing hormone (TRH) was also conjugated to NPs and DOX conjugated NPs. Briefly, in one exemplary preparation, nanogold colloids (Sigma, St. Louis, Mo.) coated with the surfactant tannic acid were first adjusted to pH 9.0 using a 0.1 M sodium carbonate solution in a silicone coated glass bottle. 0.5 mg of LHRH or TRH (Sigma, St. Louis, Mo.) was dissolved in H₂O (1.0 mg/ml) and 0.5 mg of DOX (Sigma, St. Louis, Mo.) was dissolved in H₂O (1 mg/ml), and were immediately transferred in a 500: 1 ratio to the solution of gold colloid. The mixture was then diluted to 5.0 ml with H₂O and was intensely vibrated at 4° C. for 1 hr. During incubation, tannic acid on the NPs surface was replaced with LHRH or TRH, and DOX. Gold Nanoparticles conjugated with LHRH and DOX (LHRH-NP-DOX; FIG. 1) or conjugated with TRH and DOX (TRH-NP-DOX) were subsequently formed. Unconjugated LHRH and DOX were removed from the conjugated particles by dialyzing the mixture extensively using Float-A-Lyzer® Cellulose Ester Membrane. For controls, DOX, LHRH, or TRH was also conjugated to the NPs as described above.

To determine the uptake of LHRH-NP-DOX by cancer cells and their distribution in tumors and other tissues in vivo, FluoroNanogold™-streptavidin-Alexa Fluor® 594 gold NPs (FNPs; Nanoprobes, Yaphank, N.Y.) were also conjugated to LHRH and DOX according to the procedure described above. The concentration of LHRH and DOX conjugated to the NPs or the FNPs was then measured using a spectrophotometer at OD₂₈₀ (for the LHRH peptide) and Ex480 nm/Em620 nm (for DOX), respectively. The zeta potential and size of the unconjugated and conjugated particles was measured by a direct scattering light (DSL) device using a Zeta Sizer Series, Nano-ZS, Malvern instrument. The zeta potential and size of the particles are shown in Table 1. The conjugated particles were between 100 to 200 nm in size with a negative zeta potential. The size of the conjugated particles was also in acceptable range (less than or equal to 200 nm). Approximately, 201 molecules of [D-Trp⁶] LHRH and 75 molecules of DOX were found to be conjugated to one gold NP following the methods described above. However, it is observed that by modulating the ratio of [D-Trp⁶] LHRH to DOX in the reaction mixture, conjugated nanoparticles can be obtained that display varying numbers of conjugated [D-Trp⁶] LHRH or DOX molecules.

TABLE 1
Measurement of zeta potential and size of conjugated nanoparticles.

| Conjugate | Zeta Potential (mv) | Mean size (nm) |
|---|---|---|
| NP | −17.7 | 67 (90%) |
| DOX-NP | −2.5 | 150 (90%) |
| LHRH-NP | 0 | 185 (93%) |
| TRH-NP | −20.4 | 54 (63%) |
| TRH-NP-DOX | −1.67 | 96 (90%) |
| LHRH-NP-DOX | −5 | 204 (90%) |

### Example 2

### Preparation of Liposomes Incorporating LHRH and Doxorubicin

Liposomes conjugated to a plurality of ligands for an LHRH receptor and a plurality of chemotherapeutic agents are prepared by a variety of methods and by using various types of cholesterols and phospholipids. To prepare one exemplary conjugated liposome, DSPC (1,2-disrearoyl-sn-glycero-3-phosphocholine) and DSPE-PEG2000-COOH (1,2-distearoyl-sn-glycero-3-phosphoethanolamine-N-[carboxy(polyethylene glycerol)2000] is first mixed with chloroform at a ratio of 6:3:0.6 mol % (DSPC:Chol:DSPE-PEG2000). The mixture is dried to a thin film in a rotary evaporator at 53° C. The dried film is then hydrated in 300 mM manganese sulfate, freeze thawed five times, and size-reduced using high pressure extrusion through two stacked polycarbonate filters with an 80 nm pore size. The vesicle diameter is approximately 130±20 nm. The external buffer of the carrier system is then exchanged by dialyzing at 4° C. for 48 h against 100 volumes of 300 mM sucrose with buffer changes at 18 and 36 h. Doxorubicin is then conjugated (i.e., encapsulated) to the liposomes using an ionophore-mediated proton gradient (114). Conjugation is performed with 5 mM doxorubicin and 40 mM lipid in a solution containing 300 mM sucrose, 30 mM EDTA, 20 mM 2-morpholineethanesulfonic acid (pH 6.0). The divalent cation ionophore A23187 (7 μM final concentration) is added to the liposomes, and the mixture is incubated at 65° C. for 1 h. The extent of encapsulation is determined by passing an aliquot of the sample through a SEPHADEX® G-50 spin column and the concentration of lipid and doxorubicin is measured. Uncapsulated doxorubicin and A23187 is removed from the preparation by dialyzing the sample at 4° C. for 48 h against 100 volumes of 300 mM sucrose.

Conjugation of LHRH to the liposomes surface is achieved by adding 360 μl of both EDC [N-(3-dimethylaminopropy)-N′-ethylcabodiimide hydrochloride] (0.5 M in H₂O ) and S-NHS (sulpho-N-hydroxysuccinimide) (0.5 M in H₂O ) per 10 μl of lipid, before adjusting to a pH 5.2 with citric acid. Excess EDC and S-NHS is removed by dialysis. After adjusting to a pH of 7.5 with 1 M NaOH, 125 μg LHRH/μmol PL is then added and gently stirred for 48 h at 4° C. Unbound peptide is removed by passing the liposome suspension through a SEPHADEX® CL-4B column. The concentration of bound LHRH is then measured.

### Example 3

### Binding and Biological Activity of Gold Nanoparticles Conjugated with LHRH and DOX

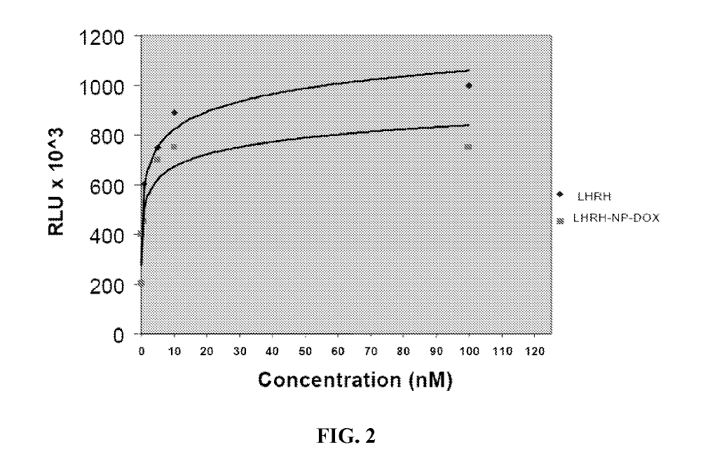

To determine if LHRH, when conjugated to gold particles or FNPs, retained its binding affinity to the luteinizing hormone-releasing hormone receptor and its biological activity, the binding of LHRH, LHRH-NP-DOXs, and LHRH-FNP-DOXs to cell surface LHRH receptors was first examined For this purpose, a mouse gonadotrope tumor cell line (LβT2) was used that expresses high levels of high affinity LHRH receptors (82). The LβT2 cells were plated on 6-well plates. After 24 hrs, the cells were transfected with a reporter gene construct, pCRE-luciferase plasmid, as described previously (82). After 24 hrs of transfection, the medium was replaced with serum free medium, followed by incubation for 4 hrs, and then treatment of the cells with various concentrations of LHRH or LHRH-NP-DOXs for 6 hrs. Cells were then lysed and assayed for luciferase activity using a luciferase assay kit (Promega, Madison, Wis.). Binding of LHRH or LHRH-NP-DOX particles was calculated and compared as described previously (82). Experiments were repeated three times for every new preparation. The binding affinities (IC₅₀ values) for LHRH and LHRH-NP-DOX were comparable (0.1 nM) (FIG. 2), indicating that the LHRH conjugated gold NPs retained a normal affinity for the receptor. Similar results were also obtained when LHRH and DOX were conjugated to FNP.

### Example 4

### Treatment of Cancer Cells with LHRH and DOX Gold Nanoparticles

To determine the effect of LHRH-NP-DOXs on tumor cell death and cell viability, three ovarian epithelial tumor cells lines (A2780, CAOV3, and UCI), and a cisplatin-resistant cell line (A2780/CP70) that expresses LHRH receptors were first treated with various concentrations of LHRH, LHRH-NPs, free DOX, DOXIL®, DOX-NPs, LHRH-NP-DOX, TRH-NPs, or TRH-NP-DOXs for 24 to 72 hrs, and the cell viability was measured at predetermined time points using a 96-titer kit (Promega, Madison, Wis.).

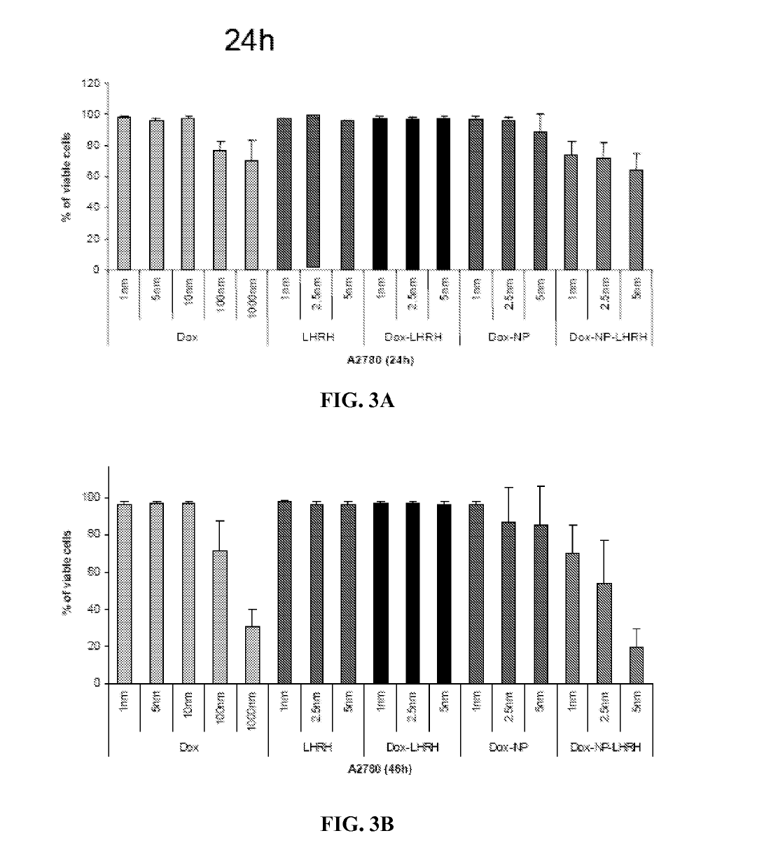

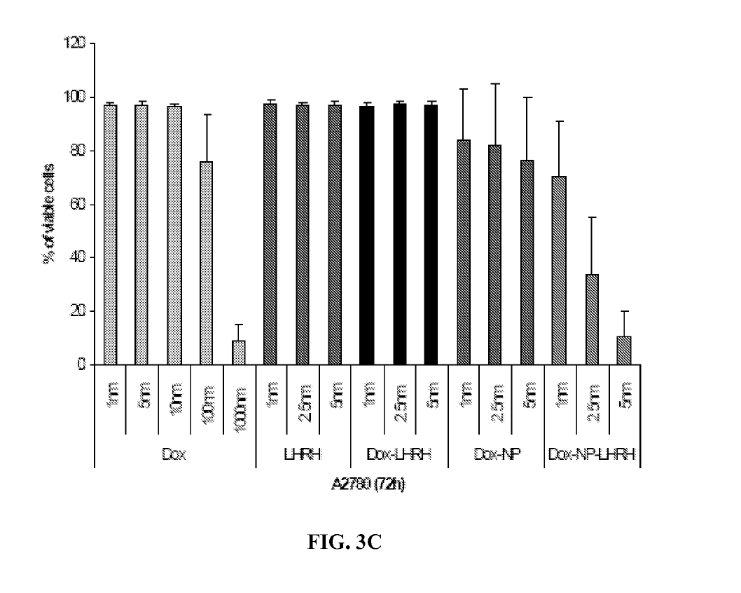

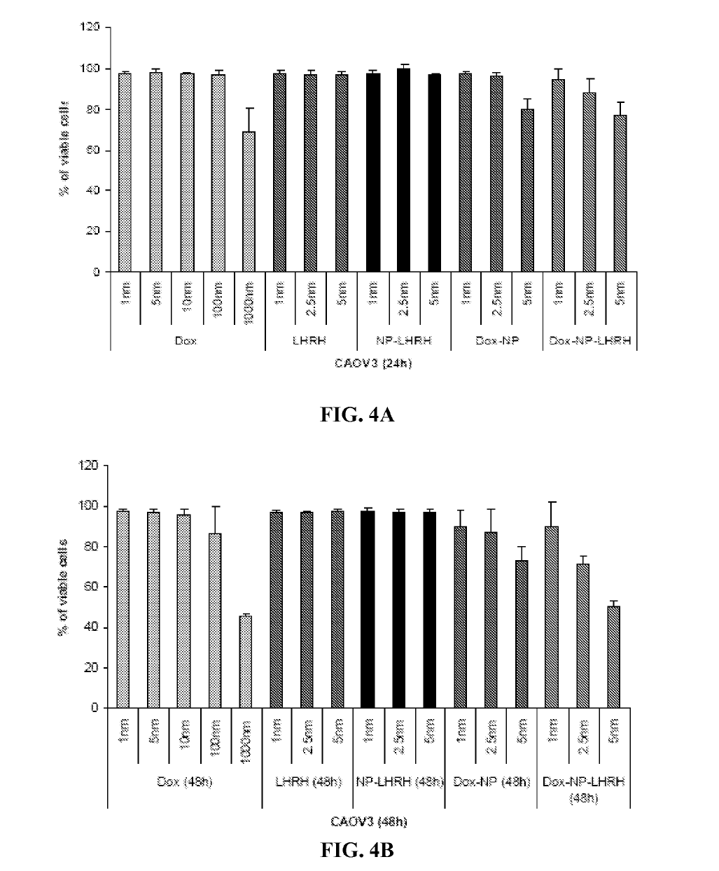

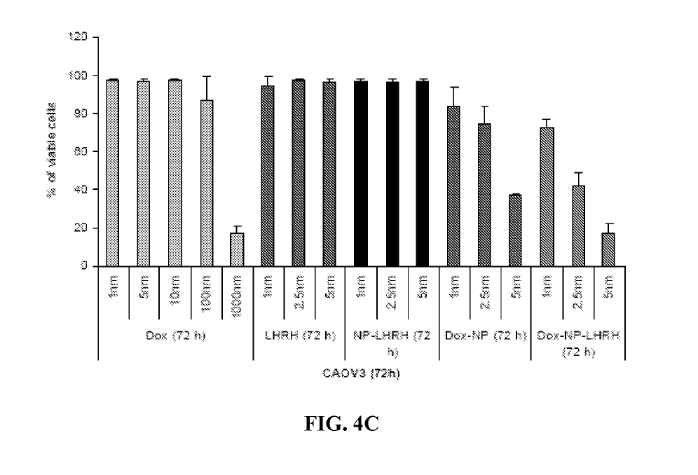

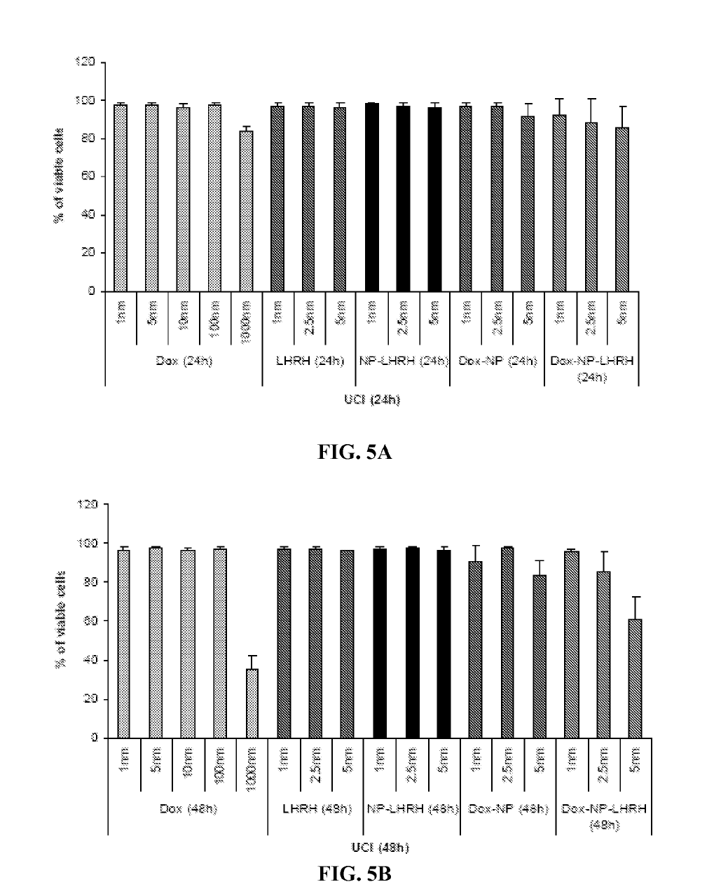

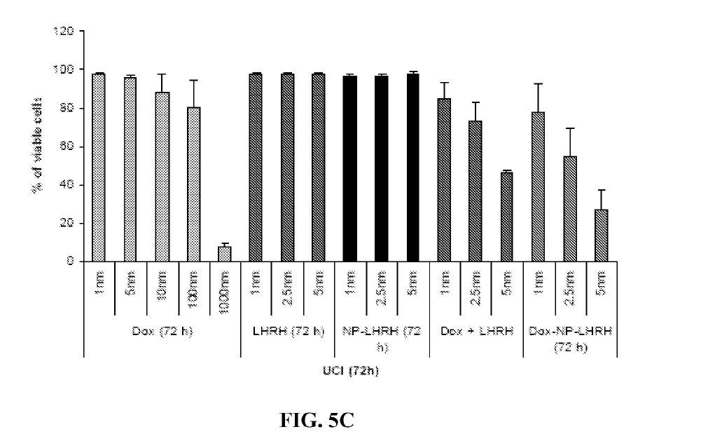

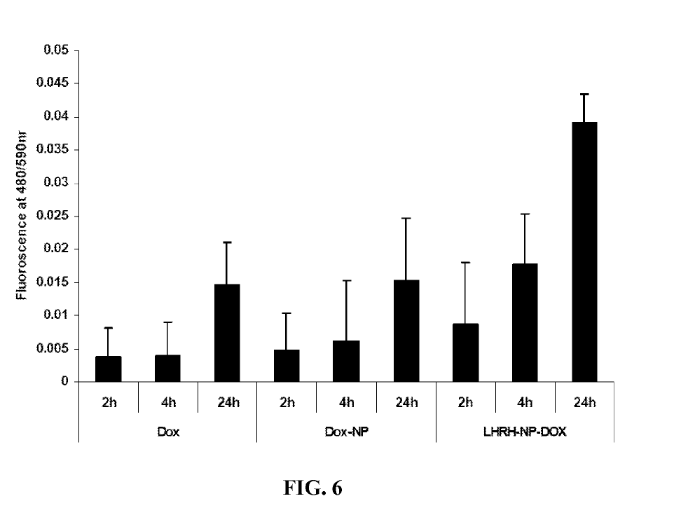

Briefly, cells from each cell line were plated in 96-well plates (3000 cells/well). After 24 hrs of plating, cells were treated with various concentrations of free DOX, DOXIL®, LHRH, LHRH-NPs, DOX-NPs, LHRH-NP-DOX, or TRH-NP-DOX (Table 2). After 24, 48, and 72 hrs of treatment, cell viability was measured by adding 20 μl of MTT reagent from the kit as described previously (83), followed by incubation at 37° C. for 2 hrs. The color developed was measured at 495 nm. Experiments were repeated three times for each cell line. As shown in FIGS. 3A-C (A2780 cells), 4A-C (CAOV3 cells), and 5A-C (UCI cells), treatment of the cells with LHRH-NP-DOX showed a dose-dependent and time-dependent cell death. After 72 hrs, treatment of the cells with 5 nM of LHRH-NP-DOX (DOX concentration) resulted in approximately 90 to 95% cell death compared to untreated cells, cells treated same concentration of free DOX, DOXIL®, DOX-NPs or LHRH, LHRH-NPs, or TRH-NP-DOX. The CAOV3 and UCI ovarian tumor cell lines showed comparatively lower cell death with equivalent amounts of LHRH-NP-DOX compared to the A2780 cell line, which could be due to lower levels of expression of LHRH receptors in these cell lines compared to A2780. Both CAOV3 and UCI cell lines, when treated with DOX-NPs, showed approximately 40-50% cell death after 72 hrs of treatment with a 5 nM concentration of DOX as compared to controls. Unconjugated DOX, DOXIL®, LHRH-NPs, LHRH, LHRH-DOX, TRH-NPs, or TRH-NP-DOX at concentrations from 1 to 5 nM were found to be ineffective for both cisplatin-sensitive and cisplatin-resistant cell lines.

TABLE 2
Efficacy of Various Conjugates.

| Grp | Conjugate | Function | Doses | Cell Death |
|---|---|---|---|---|
| 1 | PBS | Control | — | — |
| 2 | NPs | Control | — | — |
| 3 | TRH-NPs | Test | 5, 10 or 100 nm | — |
| 4 | TRH-NP-DOX | Test | 1, 2.5, 5 nM | — |
| 5 | [D-TrP⁶]LHRH | Test | 5, 10, 100 nM | — |
| 6 | LHRH-NPs | Test | 5, 10, 100 nM | — |
| 7 | DOX | Test | 1, 5, 10, 100, 1000 nM | +++ |
| | | | | (1000 nM) |
| 8 | DOXIL | Test | 1, 5, 10, 100, 1000 nM | +++ |
| | | | | (1000 nM) |
| 9 | DOX-NPs | Test | 1, 2.5, 5 nM | + (5 nM) |
| 10 | LHRH-NP-DOX | Test | 1, 2.5, 5 nM | +++ |
| | | | | (5 nM) |

Doxorubicin and DOXIL® are both used as an anticancer drug for various solid tumors and have been shown to induce cell death in various cell lines at high concentrations ranging from 1 μM to 2 μM (86). To confirm this in parallel experiments, the A2780, CAOV3, and UCI ovarian tumor cell lines were treated with DOX. In separate experiments, A2780 and A2780/CP70 cell lines were also treated with DOX and DOXIL® at variable concentrations (1 nM to 1000 nM). The ovarian tumor cells, when treated with doxorubicin and DOXIL® at a 1000 nM concentration, showed approximately 80-90% cell death within 72 hrs of treatment compared to untreated cells, or cells treated with LHRH-NPs or TRH-NP-DOXs. This level of cell death was induced by a concentration of free DOX or DOXIL® (1000 nM) that was 200-fold higher than that employed in the LHRH-NP-DOX experiments (DOX concentration=5 nM). These results indicate that conjugation of doxorubicin to LHRH-NPs selectively induces cell death as a result of an increased amount of doxorubicin being delivered to cancer cells through the LHRH receptors, which are present on the cell membranes of the ovarian tumor cell lines. The results further indicate that the targeted therapy is applicable for both cisplatin-sensitive (A2780) as well as cisplatin-resistant (A2780/CP70) cell lines, and is thus capable of being used for both cisplatin-sensitive and cisplatin-resistant cancers. Application of this targeted therapy can also minimize the chances of ovarian tumor metastasis, which typically happens after the ovarian tumors become resistant to cisplatin.

To further examine the effect of LHRH-NP-DOXs on tumor cell death and cell viability in various cancers, experiments are performed using a variety of cancer cell lines that express LHRH receptors. Briefly, the breast cancer cell lines MCF-7 and MDA-MB231, the prostate cancer cell lines Du145, and PC-3, the endometrial cancer cell lines Hec1A and Hec1B, the pancreatic cancer cell lines AR42J and ARIP, the melanoma cell lines BLM and Me15392, the testicular cancer cell lines I-10 and TCam-2, the pituitary cancer cell line LβT2, the non-Hodgkin's lymphoma cell lines R1 and HT, and the renal cell carcinoma cell lines A498 and ACHN are first grown in appropriate medium. Cells growing in the log phase are then harvested and plated in 96 well plates. After 4 h of plating, the cells are treated with various concentrations of LHRH, LHRH-NPs, DOX, DOXIL, LHRH-NP-TRH, TRH-NP-DOX, or LHRH-NP-DOX for 24 h, 48 h and 72 h, and cell death and viability is determined as described herein above. Treatment of the various cells with LHRH-NP-DOX shows a dose-dependent and time-dependent cell death. An increase in cell death is also observed in cells treated with LHRH-NP-DOX as compared to untreated cells, cells treated same concentration of free DOX, DOXIL®, DOX-NPs, LHRH, LHRH-NPs, or TRH-NP-DOX, indicating that a method including administering a nanoparticle conjugated to a plurality of a ligands for an LHRH receptor and a plurality of a chemotherapeutic agent is useful for treating various types of cancers.

### Example 5

### Internalization of LHRH-NP-DOX and Determination of Intracellular Doxorubicin

To determine the uptake of doxorubicin by ovarian tumor cells in vitro, ovarian tumor cells (A2780) were plated in 6-well plates. After 24 hrs of incubation, the medium was replaced with fresh medium and cells were then treated with PBS, free DOX (DOX concentration=5 nM), DOX-NPs (5 nM DOX), or LHRH-NP-DOXs (5 nM DOX). After 2, 4, and 24 hrs of incubation, cells were rinsed with PBS and scrapped off the plates and centrifuged. The pellets were resuspended in lysis buffer [0.25 mM sucrose, 5 mM TrisHCl, 1 mM MgSO₄, 1 mM CaCl₂ (pH 7.6)] and homogenized using a polytron homogenizer. A 200:1 dilution of the homogenate from each sample was then transferred in triplicate to 2 ml centrifuge tubes and 100:1 of 10% (v/v) Triton X-100, 200:1 of water, and 1,500:1 of acidified isopropanol (0.75 N HCl) was added. The tubes were vortexed to ensure complete mixing and doxorubicin was extracted overnight at −20° C. The next day, the samples were thawed to room temperature, and then centrifuged at 15,000×g for 20 min. The supernatant was collected. Doxorubicin was measured fluorometrically (EX_{485 nm}/EM_{590 nm}) as described previously (3).

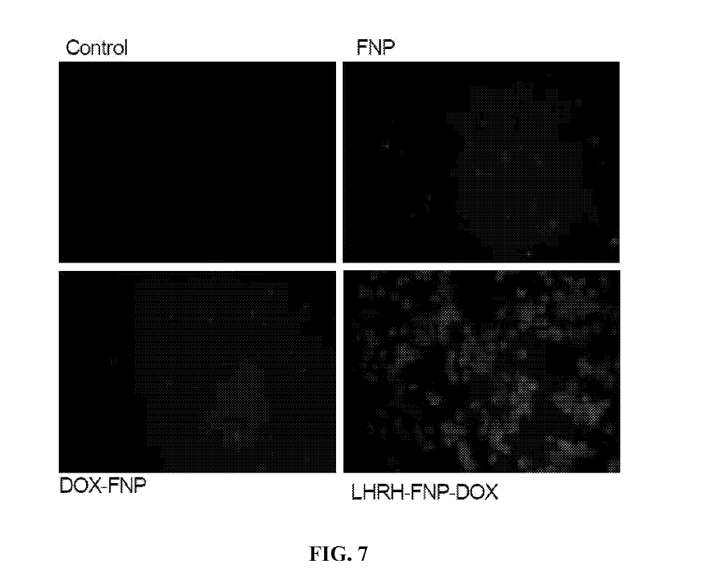

A significant (P<0.001) amount of doxorubicin was found in tumor cells treated with LHRH-NP-DOXs within 2 hrs, and this amount increased with time (4 and 24 hrs). In contrast, no detectable amount of doxorubicin was found in cells treated with free DOX or DOX-NPs within 4 hrs. However, some amount of doxorubicin was found after 24 hrs in these cells, however, it was significantly lower compared to cells treated with LHRH-NP-DOXs (FIG. 6), indicating that uptake of LHRH-NP-DOXs was specific and required conjugation of LHRH for doxorubicin delivery to the tumor cells.

To further confirm the ability of LHRH conjugated NPs to deliver doxorubicin to the ovarian tumor cells, and to determine the internalization and distribution of LHRH-NP-DOXs to the sub-cellular organelles, ovarian tumor cells (A2780) were plated in 6-well chamber slides. After 24 hrs of plating, medium was replaced with fresh growth medium followed by treatment of cells with PBS, FNPs, DOX-FNPs (final concentration of DOX=5 nM), or LHRH-FNP-DOXs (final concentration of DOX=5 nM). The concentration of the FNPs was adjusted to the concentration of the conjugated FNP. After 2, 4, and 24 hrs of incubation, medium was aspirated and cells were washed twice with PBS. The cells were examined under a fluorescence microscope (Olympus X50) and photographed. Low or undetectable levels of internalization of conjugated particles were observed after 2 hrs of incubation. A moderate level was observed after 4 hrs of incubation and most of the FNP particles were found to be present near the plasma membrane and in cytoplasm. After 24 hrs of incubation, a large amount of conjugated particles were internalized into tumor cells and were translocated to the cytoplasm, internal organelles, and nucleus. In contrast, no or a negligible amount of particles was found in tumor cells upon treatment of the cells with unconjugated FNP or conjugated DOX-FNPs (FIG. 7). These results again confirm that uptake of LHRH-NP-DOX particles by ovarian tumor cells is highly specific and is mediated through the high affinity LHRH receptors present on the cell membrane.

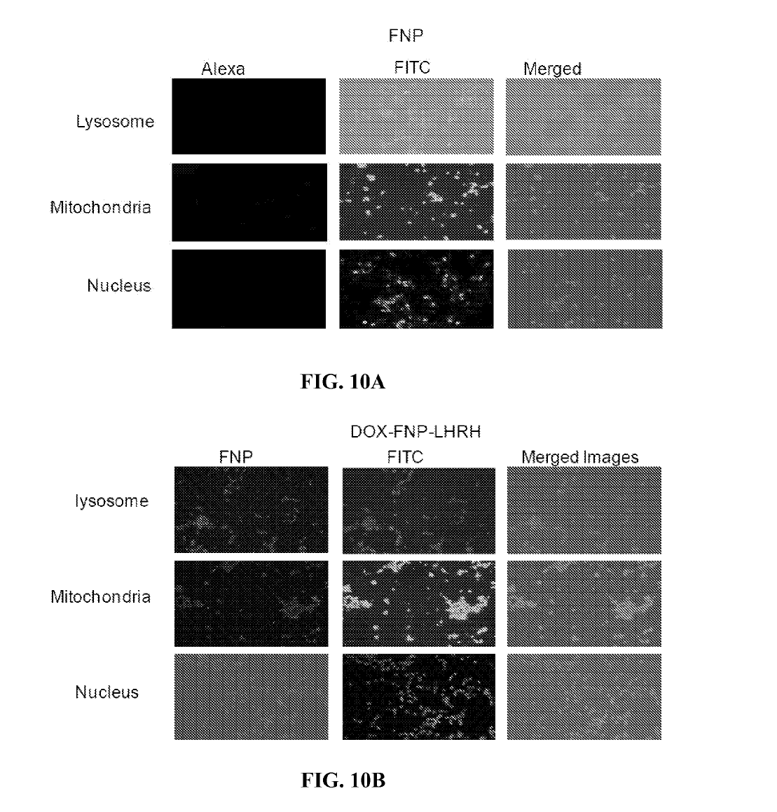

To determine the time-based translocation of LHRH-FNP-DOX to specific organelles, specific biomarkers for each organelle (mitochondria, lysosomes, and nucleus) were used. Colocalization of the biomarker and FNPs showed translocation of particles to the lysosomes, mitochondria, and nucleus of the cells, with a large amount of nanoparticles being observed in the lysosomes and mitochondria (FIGS. 10A and 10B). Comparatively, a lower amount of particles was observed in the nucleus, thus indicating that doxorubicin is released from the conjugates in the lysosomes and is then translocated to nucleus, while the nanoparticles themselves remained distributed in the lysosomes and mitochondria.

### Example 6

### Effects of LHRH- and Doxorubicin-Conjugated Gold NPs on Tumor Growth In Vivo

To assess the effect of LHRH-NP-DOX particles on tumor growth, their specificity, and their distribution in tumors and other tissues in vivo, two models were generated: i) a subcutaneous xenograft mouse model, and ii) a mouse intraperitoneal ovarian tumor model. For the subcutaneous xenograft tumor model, epithelial ovarian tumor cells (A2780) growing in log phase were trypsinized, washed twice with PBS, and resuspended in serum and antibiotic-free Dulbecco's modified eagle medium (DMEM; 20×10⁶/ml). 100 μl of these cells (2×10⁶/site) were injected subcutaneously into both flanks of 5- to 6-week-old female nu/nu mice (Charles River) as described previously (83). The animals were examined for the development of visible subcutaneous tumors. For the intra-peritoneal ovarian tumor model, a 500:1 cell suspension (10×10⁶cells/ml) was injected intra-peritoneally into each nu/nu female mouse (5 to 6 weeks of age). Two weeks after the injection of the cells, the animals were examined for the development of intra-peritoneal tumors, and metastasis to other organs such as ovaries, lung, liver, uterus, and intestine, using a stereo microscope. Two weeks after the injection of the tumor cells, the animals showed development of visible tumors in both the subcutaneous-injected and intra-peritoneal-injected groups.

The animals were then divided into six groups (5 animals/group) and were injected intra-peritoneally with 200:1 dilutions of PBS (Group 1), free DOX (Group 2, final concentration of doxorubicin 9.6 μg/Kg), DOX-NPs (Group 3, final concentration of doxorubicin 9.6 μg/Kg), LHRH-NP-DOX (Group 4, final concentration of doxorubicin 9.6 μg/Kg), or LHRH-FNP-DOXs (Group 5, final concentration of doxorubicin 9.6 μg/Kg). A total of four injections were given. The first three injections were given at three days intervals, and the fourth was given two days after the third injection (due to large tumors in controls). Before each injection, tumor volumes (mm³) were measured according to the formula: tumor volume (mm³)=(long diameter)×(short diameter)²/2. Three days after the last injection, the animals were sacrificed, and the tumors were collected and weighed. One part of the tumor and other tissues were fixed in 10% buffered formalin and another part was frozen in liquid nitrogen and stored at −80° C. for measurement of intracellular free DOX and for future use. All animal studies were carried out in accordance with the University of Louisville Institutional Animal Care and Use Committee Guidelines.

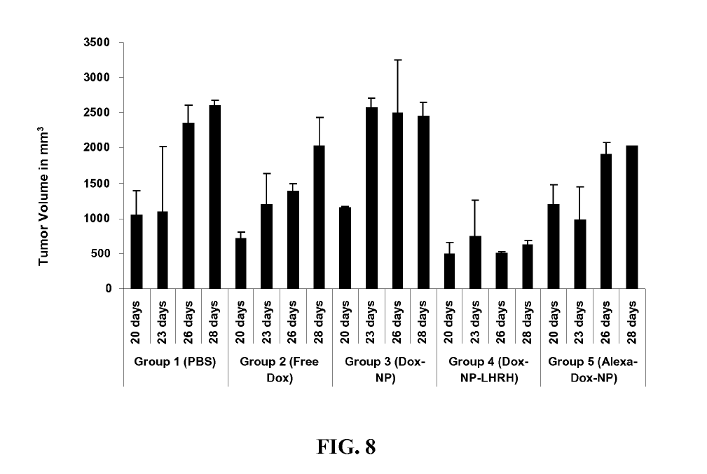

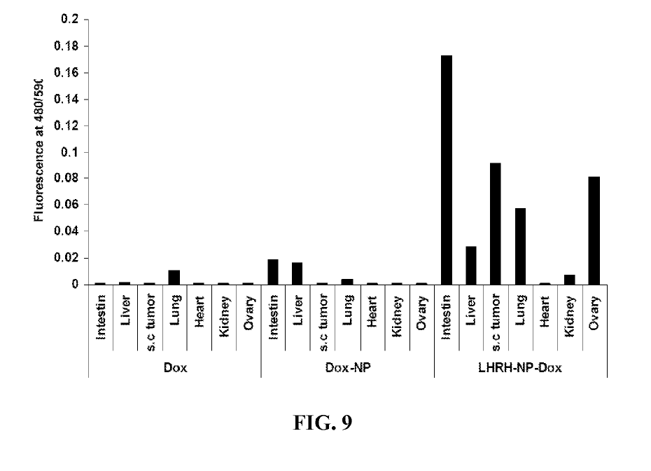

Gross analysis of intra-peritoneal-injected animals showed aggressive growth of intra-peritoneal tumors and metastasis to intestines, ovaries, lungs, and uterus. There appeared to be reduced growth and metastasis in animals injected with LHRH-NP-DOXs compared to animals injected with PBS, DOX-NPs, or free DOX. In subcutaneous-injected animals, growth of tumors was significantly (P<0.001) suppressed in animals treated with LHRH-NP-DOXs compared to animals treated with PBS, DOX-NPs, or free DOX (FIG. 8). There was also a reduction in tumor growth in animals injected with LHRH-FNP-DOXs compared to animals injected with PBS or free DOX-NPs. However, suppression of tumor growth in these animals was lower compared to animals injected with LHRH-NP-DOXs. Measurement of intracellular DOX in intra-peritoneal and subcutaneous tumors as well as in metastatic tumors showed a significantly (P<0.001) higher accumulation of DOX in primary tumors or metastatic tumors collected from animals treated with LHRH-NP-DOXs compared to animals treated with free DOX or DOX-NPs (FIG. 9). Further, because DOX is known to cause myocardial toxicity, intracellular levels of DOX was also measured in heart tissues. No detectable amount of DOX was found in the heart of these animals. Similarly, no doxorubicin or undetectable amounts were observed in other normal or non-metastatic tissues such as the kidney or oviduct.

The dose of doxorubicin or its liposome prep (DOXIL®) used by others (3, 86) in subcutaneous or intra-peritoneal tumor models varies from 9.0 mg/kg to 16 mg/kg, which results in cytotoxicity to normal tissues and organs including myocardial toxicity. In the presently-described experiments, 9.6 μg/kg of doxorubicin was used, which is approximately 1000-fold lower compared to free DOX used by others. These results thus indicate that by conjugating LHRH and doxorubicin to nanogold particles, significantly lower doses of doxorubicin are able to be used. By specifically targeting cancer cells with LHRH, a high payload of doxorubicin, which is required to induce cancer cell death, can thus be delivered to cancer cells while avoiding toxicity to normal cells and tissues.

To further examine the ability of LHRH-NP-DOXs on tumor growth, their specificity, and their distribution in tumors and other tissues in vivo, in vivo models for a variety of different cancer types are generated. Briefly, cells from the breast cancer cell lines MCF-7 and MDA-MB231, the prostate cancer cell lines Du145 and PC-3, the endometrial cancer cell lines Hec1A and Hec1B, the pancreatic cancer cell lines AR42J and ARIP, the melanoma cell lines BLM and Me15392, the testicular cancer cell lines I-10 and TCam-2, the pituitary cancer cell line LβT2, the non-Hodgkin's lymphoma cell lines R1 and HT, and the renal cell carcinoma cell lines A498 and ACHN are injected subcutaneously into nu/nu mice. Two weeks after the injection of the cancer cells, visible tumors develop and the mice are divided into several groups. The mice are then treated with various un-conjugated and conjugated nanoparticles as described above. Treatment of the various models with LHRH-NP-DOX induces cell death, suppresses tumor growth, and decreases metastasis in vivo. Further, the DOX dosage administered via LHRH-NP-DOX to suppress cell proliferation and tumor growth in vivo is lower than free DOX or DOXIL®, indicating that a method including administering a nanoparticle conjugated to a plurality of a ligands for an LHRH receptor and a plurality of a chemotherapeutic agent is useful for treating various types of cancers, and that lower doses of a chemotherapeutic agent are required to achieve cancer cell death when the chemotherapeutic agent is conjugated to a nanoparticle that is also conjugated to a plurality of ligands for an LHRH receptor.

### Example 7

### PEGylation of Conjugated Nanoparticles

To determine if PEGylation improves the quality and half-life of the conjugated particles, PEGylation of conjugated DOX-NPs, LHRH-NPs, TRH-NP-DOX, and DOX-NP-LHRH, and unconjugated gold nanoparticles is performed as described previously (95). Briefly, conjugated and unconjugated particles are added to methoxypoly(ethylene glycol) (mPEG) (Mᵣ. 2 kDa) conjugated to cyanuric chloride suspended in isotonic alkaline PBS (50 mM K₂HPO₄/105 mM NaCl, PH˜9.2) to a final concentration of 1 μM of mPEG. The mixture is then incubated for 30 min at 4° C. The final PEGylated nanocomposite particles are placed in 14,000 MW cutoff dialysis bags in a 1 L PBS reservoir at 4° C., and are changed twice daily for one week (96). The particles are then analyzed for their zeta potential and size measurement using a direct scattering light (DSL) technique (Zeta Sizer Series, Nano-ZS, Malvern Instruments). Electron microscopic analysis of the unPEGlylated and PEGylated particles is performed to confirm the size of the particles (96). LHRH and doxorubicin is also measured as described herein above.

To determine the binding affinities of unPEGylated and PEGylated LHRH, LHRH-NP-DOX, and LHRH-NPs to LHRH receptors and the activation of LHRH receptors, radioreceptor assays (20) as well as measurements of intracellular IP₃ levels (82) are conducted. The radioreceptor assays and the measurement of intracellular IP₃ levels show that PEGylation of nanoparticles conjugated with LHRH and DOX does not significantly alter the binding affinity and biological activity of LHRH for its receptor, indicating that PEGylated nanoparticles can effectively be used without affecting the binding affinity and biological activity of LHRH.

To determine whether PEGylation can effectively be used to increase the circulation time and half-life of DOX, in vivo tumor models are generated by injecting tumor cells subcutaneously into nu/nu mice as described above. After the mice develop visible tumors, groups of mice are then treated with unPEGylated or PEGylated LHRH-NP, DOX-NP or LHRH-NP-DOX. Pharmacokinetic analysis of DOX in plasma, tumors, and other tissues is then performed each day for 15 days after the treatment. An enhanced half life and retention of molecules in tumors for longer periods of time is observed for PEGylated LHRH-NP-DOX as compared to unPEGylated LHRH-NP-DOX, indicating that PEGylation can effectively be used to increase the circulation time and half-life of LHRH-NP-DOX in vivo.

Throughout this document, various references are mentioned. All such references are incorporated herein by reference, including the references set forth in the following list:

### REFERENCES

1. Maeda H, Wu J, Sawa T, Matsumura Y, Hori K. Tumor vascular permeability and the EPR effect in macromolecular therapeutics: a review. J Control Release. 65: 271-84, 2000.
2. Maeda H, Sawa T, Konno T. Mechanism of tumor-targeted delivery of macromolecular drugs, including the EPR effect in solid tumor and clinical overview of the prototype polymeric drug SMANCS. J Control Release. 74: 47-61, 2001.
3. Laginha K M, Verwoert S, Charrois G J, Allen T M. Determination of doxorubicin levels in whole tumor and tumor nuclei in murine breast cancer tumors. Clin Cancer Res 11: 6944-6949, 2005.
4. Wildiers H, Jurcut R, Ganame J, Herbots L, Neven P, De Backer J, Denys H, Cocquyt V, Rademakers F, Voigt J U, Paridaens R A. Pilot study to investigate the feasibility and cardiac effects of pegylated liposomal doxorubicin (PL-DOX) as adjuvant therapy in medically fit elderly breast cancer patients. Crit Rev Oncol Hematol 67: 133-138, 2008.
5. Abuchowski A, van Es T, Palczuk N C, Davis F F. Alteration of immunological properties of bovine serum albumin by covalent attachment of polyethylene glycol. J Biol Chem 252: 3578-3581, 1977.
6. Pohlgeers K M, Panguluri S K, Kakar S S. Implication of nanoparticles in diagnosis and treatment of cancer: the future of oncology. Cell death signaling and cancer 2009: 145-147.
7. Singh R, Lillard J W Jr. Nanoparticle-based targeted drug delivery. Exp Mol Pathol 2009. In press.
8. Jemal A. Siegel R. Ward E. Hao Y., Xu J., Murray T. Thun M J. CA: Cancer statistics, 2008. CA Cancer J. Clin 58: 71-96, 2008.
9. Lin H, Changchien C C. Management of relapsed/refractory epithelial ovarian cancer: current standards and novel approaches. Taiwan J Obstet Gynecol 46: 379-388, 2007.
10. Schwartz P E. What is the role of neoadjuvant chemotherapy in the management of ovarian cancer? Oncology (Williston Park) 22: 1118-1125, 2008.
11. Patel H M. Serum opsonins and liposomes: their interaction and opsonophagocytosis. Crit Rev Ther Drug Carrier Syst. 9(1):39-90. 1992.
12. Devine D V. Marjan J M. The role of immunoproteins in the survival of liposomes in the circulation. Crit Rev Ther Drug Carrier Syst. 14(2):105-31. 1997. Review.
13. Petri-Fink A. Chastellain M. Juillerat-Jeanneret L. Ferrari A. Hofmann H. Development of functionalized superparamagnetic iron oxide nanoparticles for interaction with human cancer cells. Biomaterials 26: 2685-2694, 2005.
14. Milacic V. Fregona D, Diu Q P. Gold complexes as prospective metal-based anticancer drugs. Histo Histopathol. 23: 101-108, 2008.
15. Forestier J. Rheumatoid arthritis and its treatment with gold salts-results of six years's experience. J Lab Clin Med 20: 827-300, 1935.
16. Messori I Abbate F., Marcon G., Orioli P., Fontani M., Mini E., Mazzei T., Carotti S., O'Connell T., Zanello P. Gold(III) complexes as potential antitumor agents: solution chemistry and cytotoxic properties of some selected gold(III) compounds. J Med Chem. 43(19):3541-8, 2000.
17. Maccon G. Carotti S., Coronnello M., Messori L., Mini E., Orioli P., Mazzei T., Cinellu M A., Minghetti G. Gold(III) complexes with bipyridyl ligands: solution chemistry, cytotoxicity, and DNA binding properties. J Med Chem. 45(8):1672-7, 2002.
18. Bruni B. Guerri A., Marcon G., Messori L., Orioli P. Structure and cyctotoxic properties of some selected gold (III) complexes. Croatica Chemica Acta 72: 221-229.
19. Kakar S S. Malik M T. Winters S J. Mazhawidza W. Gonadotropin-releasing hormone receptors: structure, expression, and signaling transduction. Vit Horm 69: 151-207, 2004.
20. Kakar S S. Musgrove L C. Devor D C. Sellers J C. Neill J D. Cloning, sequencing, and expression of human gonadotropin releasing hormone (GnRH) receptor. Bioch Biophy Res Commun 189: 289-295, 1992.
21. Kakar S S. Grizzle W E. Neill J D. The nucleotide sequences of human GnRH receptors in breast and ovarian tumors are identical with that found in pituitary. Molec Cell Endocrin 106: 145-149, 1994.
22. Nagy A. Schally A V. Targeting of cytotoxic luteinizing hormone-releasing hormone analogs to breast, ovarian, endometrial, and prostate cancers. Biol Reprod 73: 851-859, 2005.
23. Zhou J. Leuschner C. Kumar C. Hormes J F. Soboyejo W O. Sub-cellular accumulation of magnetic nanoparticles in breast tumors and metastases. Biomaterials 27: 2001-2008, 2006.
24. Leuschner C, Kumar c, Urbina M, Zhou M, Zhou J, Soboyejo W, Hansel W et al. The use of ligand conjugated superparamagnetic iron oxide nanoparticles (SPION) for early detection of metastases. In: NSTI Nanotech 2005. Technical Proceedings, Vol 1. 2005. P. 5-6.
25. Norberto S, Kakar S S. The use of nanoparticles in LHRH receptor targeted therapy for cancer. Expt Mol Path 2009, In press.
26. Dharap S S. Wang Y. Chandna P. Khandare J J. Qiu B. Gunaseelan S. Sinko P J. Stein S. Farmanfarmaian A. Minko T. Tumor-specific targeting of an anticancer drug delivery system by LHRH peptide. Proc Nat Acad Sci USA 102: 12962-12967, 2005.
27. Klijn, J. M. G. and Foekens, J. A. (1989) Extrapituitary actions. In: Basic aspects. GnRH analogues in cancer and human reproduction, pp 71-84, 1998. Editors: B. H. Vickey and B. Lunenfel. Kluwar Academic Publisher.
28. Emons G. Grundker C. Gunthert A R. Westphalen S. Kavanagh J. Verschraegen C. GnRH antagonists in the treatment of gynecological and breast cancers. Endocrin-Relat Cancer 10: 291-9, 2003.
29. Eidne, K A. Flanagan, C A., Harris, N S. and Millar, R P. Gonadotropin-releasing hormone (GnRH)-binding sites in human breast cancer cell lines and inhibitory effects of GnRH antagonists. J. Clin Endocr Metab 64: 425-432, 1987.
30. Eidne, K A. Flanagan, C A. and Millar, R P. Gonadotropin releasing-hormone binding sites in human carcinoma. Science, 229, 989-991, 1985.
31. Bramley T A. McPhie C A. Menzies G S. Human placental gonadotropin-releasing hormone (GnRH) binding sites: Characterization, properties and ligand specificity. Placenta 13: 555-581, 1992.
32. Bramley T A. Menzies G S. Baird D T. Specificity of gonadotropin-releasing hormone binding sites of the human corpus luteum: comparison with receptors of rat pituitary gland. J Endocr 108: 323-328, 1986.
33. Emons G. Pahwa G S. Brack C. Sturm R. Oberhauser F. Knuppen R. Gonadotropin releasing hormone binding sites in human epithelial ovarian carcinomata. Eur J Cancer Clin Oncol 25:215-221, 1989.
34. Emons G. Schally A V. The use of luteinizing hormone releasing hormone agonists and antagonists in gynecological cancers. Hum Rep. 9: 1364-1379, 1994.
35. Henzl M R. Gonadotropin-releasing hormone and its analogues: from laboratory to bedside. Clin Obstr Gyn 36: 617-635, 1993.
36. Schally A V. Schally-Comaru A M. Redding T W. Antitumor effects of analogs of hypothalamic hormones in endocrine-dependent cancers. Proc Soc Exp Biol Med. 175: 259-281, 1984.
37. Harvey H A. Lipton A. Max D T. LH-RH and its analogues, contraceptive and clinical application, pp 329-328, 1984. MTP Press, Lancaster.
38. Klijn J G. de Jong F H. Lamberts S W. Blankenstein M A. LHRH-agonist treatment in clinical and experimental human breast cancer. J Steroid Biochem 23: 867-873, 1985.
39. Dondi D. Moretti R M. Montagnani Marelli M. Pratesi G. Polizzi D. Milani M. Motta M. Limonta P. Growth-inhibitory effects of luteinizing hormone-releasing hormone (LHRH) agonists on xenografts of the DU 145 human androgen-independent prostate cancer cell line in nude mice. Intern J Cancer. 76: 506-11, 1998.
40. Blankenstein M A. Henkelman M S. Klijn J G. Direct inhibitory effect of a luteinizing hormone-releasing hormone agonist on MCF-7 human breast cancer cells. Eur J Cancer Clin Oncol 21: 1493-1499, 1985.
41. Miller W R. Scott W N. Morris R. Fraser H M. Sharpe R M. Growth of human breast cancer cells inhibited by a luteinizing hormone-releasing hormone agonist. Nature. 313: 231-233, 1985.
42. Eidne K A. Flanagan C A. Harris N S. Millar R P. Gonadotropin-releasing hormone (GnRH)-binding sites in human breast cancer cell lines and inhibitory effects of GnRH antagonists. J Clin Endocrin Metab 64: 425-32, 1987.
43. Fekete, M., Wittliff, J L. Schally, A V. Characterization and distribution of receptors for [D-Trp⁶]-luteinizing hormone-releasing hormone, somatostatin, epidermal growth factor, and sex steroid in 500 biopsy samples of human barest cancer. J Clin Lab Anal 3: 137-147, 1989.
44. Baumann K H. Kiesel L. Kaufmann M. Bastert G. Runnebaum B. Characterization of binding sites for a GnRH-agonist (buserelin) in human breast cancer biopsies and their distribution in relation to tumor parameters. Breast Cancer Res Treat 25: 37-46, 1993.
45. Keri G. Balogh A. Szoke B. Teplan I. Csuka O. Gonadotropin-releasing hormone analogues inhibit cell proliferation and activate signal transduction pathways in MDA-MB-231 human breast cancer cell line. Tumour Biol 12(2):61-67, 1991.
46. Emons G. Pahwa G S. Brack C. Sturm R. Oberhauser F. Knuppen R. Gonadotropin releasing hormone binding sites in human epithelial ovarian carcinomata. Eur J Cancer Clin Oncol 25: 215-221, 1989.
47. Pahwa G S. Vollmer G. Knuppen R. Emons G. Photoaffinity labeling of gonadotropin releasing hormone binding sites in human epithelial ovarian carcinomata. Bioch Biophys Res Commun 161: 1086-1092, 1989.
48. Emons G., Schroder, B. Ortmann, O. Westphalen S. Schulz K-D. Schally, A V. (1993) High affinity binding and direct antiproliferative effects of luteinizing hormone-releasing hormone analogs in human endometrial cancer cell lines. J. Clin Endocr Metab 77: 1458-1464, 1993.
49. Emons G. Ortmann O. Becker M. Irmer G. Springer B. Laun R. Holzel F. Schulz K-D. Schally A V. High affinity binding and direct antiproliferative effects of LHRH analogues in human ovarian cancer cell lines. Cancer Res., 53, 5439-5446, 1993.
50. Thompson M A. Adelson M D. Kaufman L M. Lupron retards proliferation of ovarian epithelial tumor cells cultured in serum-free medium. J Clin Endocrin Metab 72: 1036-41, 1991.
51. Yano T. Pinski J. Radulovic S. Schally A V Inhibition of human epithelial ovarian cancer cell growth in vitro by agonistic and antagonistic analogues of luteinizing hormone-releasing hormone. Proc. Natl. Acad. Sci. USA, 91, 1701-1705, 1994.
52. Pahwa G S. Kullander S. Vollmer G. Oberheuser F. Knuppen R. Emons G. Specific low affinity binding sites for gonadotropin-releasing hormone in human endometrial carcinomata. Eur J Obst Gyn Reprod Biol 41: 135-142, 1991.
53. Imai A. Ohno T. Lida K. Fuseya T. Furui T. Tamaya T. Presence of gonadotropin-releasing hormone receptor and its messenger ribonucleic acid in endometrial carcinoma and endometrium. Gynec Onco155: 114-148, 1994.
54. Grundker C. Gunthert A R. Westphalen S. Emons G. Biology of the gonadotropin-releasing hormone system in gynecological cancers. Eur J Endocrin 146: 1-14, 2002.
55. Vincze B. Palyi I. Daubner D. Kremmer T. Szamel I. Bodrogi I. Sugar J. Seprodi J. Mezo I. Teplan I. et al. Influence of luteinizing hormone-releasing hormone agonists on human mammary carcinoma cell lines and their xenografts. J steroid Biochem Molec Biol 38: 119-26, 1991.
56. Pati D. Habibi H R Inhibition of human hepatocarcinoma cell proliferation by mammalian and fish gonadotropin-releasing hormones. Endocrinology, 36, 75-84, 1995.
57. Fekete M. Bajusz S. Groot K. Csernus V J. Schally A V. Comparison of different agonists and antagonists of luteinizing hormone-releasing hormone for receptor-binding ability to rat pituitary and human breast cancer membranes. Endocrinology, 124, 946-955, 1989.
58. Fekete M. Redding T W. Comaru-Schally A M. Pontes J E. Connelly R M. Srkalovic G. Schally A V. Receptors for luteinizing hormone-releasing hormone, somatostatin, prolactin, and epidermal growth factor in rat and human prostate cancers and in benign prostate hyperplasia. Prostate 14: 191-208, 1989.
59. Chen A. Kaganovsky E. Rahimipour S. Ben-Aroya N. Okon E. Koch Y. Two forms of gonadotropin releasing hormone (GnRH) are expressed in human breast tissue and overexpressed in breast cancer: a putative mechanism for the antiproliferative effect of GnRH by down-regulation of acidic ribosomal phosphoproteins P1 and P2. Cancer Res. 62:1036-1044, 2002.
60. Li L-S. Roberts V J. Yen S S. Expression of human gonadotropin-releasing hormone receptor gene in the placenta and its functional relationship to human chorionic gonadotropin secretion. J Clin Endocrinol Metab 80: 580-585, 1995.
61. Dondi D. Limonta P. Moretti R M. Marelli M M. Garattini E. Motta M. Antiproliferative effects of luteinizing hormone-releasing hormone (LH RH) agonists on human androgen-independent prostate cancer cell line DU 145: evidence for an autocrine-inhibitory LH RH loop. Cancer Res 54: 4091-5, 1994
62. Limonta P. Dondi D. Moretti R M. Maggi R. Motta M. Antiproliferative effects of luteinizing hormone releasing hormone agonists on the human prostatic cancer cell line LNCaP. J Clin Endocrin Metab 75: 207-212, 1992.
63. Chatzaki E. Bax C M. Eidne K A. Anderson L. Grudzinskas J G. Gallagher C J. The expression of gonadotropin-releasing hormone and its receptor in endometrial cancer, and its relevance as an autocrine growth factor. Cancer Res 56: 2059-2065, 1996.
64. Halmos G. Schally A V. Kahan Z. Down-regulation and change in subcellular distribution of receptors for luteinizing hormone-releasing hormone in OV-1063 human epithelial ovarian cancers during therapy with LH-RH antagonist Cetrorelix. Intern J Oncol 17: 367-73, 2000.
65. Irmer G. Burger C. Muller R. Ortmann O. Peter U. Kakar S S. Neill J D. Schulz K-D. Emons G. Expression of the messenger RNAs for luteinizing hormone-releasing hormone (LHRH) and its receptor in human ovarian epithelial carcinoma. Cancer Res., 55, 817-822, 1995.
66. Kottler M-L. Starzec A. Carre M-C. LaGarde J-P. Martin A. Counis R. The genes for gonadotropinreleasing hormone and its receptor are expressed in human breast with fibrocystic disease and cancer. Int J Cancer 71: 595-599, 1997.
67. Ohno T. Imai A. Furui T. Takahashi K. Tamaya T. Presence of gonadotropin-releasing hormone and its messenger ribonucleic acid in human ovarian epithelial carcinoma. Am J Obst Gyn 169: 605-610, 1993.
68. Yin H. Cheng K W. Hwa H L. Peng C. Auersperg N. Leung PC. Expression of the messenger RNA for gonadotropin-releasing hormone and its receptor in human cancer cell lines. Life Sci 62: 2015-2023, 1998.
69. Limonta P. Dondi D. Moretti R M. Fermo D. Garattini E. Motta M. Expression of luteinizing hormone mRNA in the human prostatic cancer cell line LNCaP. J Clin Endocrin Metab 76: 797-800, 1993.
70. Minaretzis D. Jakubowski M. Mortola J F. Pavlou S N. Gonadotropin-releasing hormone receptor gene expression in human ovary and granulosa-lutein cells. J Clin Endocrin Metab 80: 430-434, 1995.
71. Bahk J Y. Hyun J S. Lee J Y. Kim J Cho Y H. Lee J H. Park J S. Kim M O. Concentration of ofloxacin in canine prostate tissue and prostate fluid after intraprostatic injection of biodegradable sustained releasing microspheres containing ofloxacin. J Urol 163: 1560-1564, 2000.
72. Halmos G. Arencibia J M. Schally A V. Davis R. Bostwick D G. High incidence of receptors for luteinizing hormone-releasing hormone (LHRH) and LHRH receptor gene expression in human prostate cancers. J Urol 163: 623-629, 2000.
73. Straub B. Muller M. Krause H. Schrader M. Goessl C. Miller K. Receptor gene messenger RNA expression in metastatic lesions of prostate cancer. J Urol 168: 1212-1214, 2002.
74. Tieva A. Stattin P. Wikstrom P. Bergh A. Damber J E. Gonadotropin-releasing hormone receptor expression in the human prostate. Prostate 47: 276-284, 2001.
75. Wolfe A. Kim H H. Radovick S. The GnRH neuron: molecular aspects of migration, gene expression and regulation. Prog Brain Res 141: 243-57, 2002.
76. Mangia A. Tommasi S. Reshkin S J. Simone G. Stea B. Schittulli F. Paradiso A. Gonadotropin releasing hormone receptor expression in primary breast cancer: comparison of immunohistochemical, radioligand and Western blot analyses. Oncol Rep 9: 1127-32, 2002.
77. Imai A. Ohno T. Iida K. Fuseya T. Furui T. Tamaya T. Presence of gonadotropin-releasing hormone receptor and its messenger ribonucleic acid in endometrial carcinoma and endometrium. Gyn Oncol 55: 144-148, 1994.
78. Nagai N. Oshita T. Mukai K. Shiroyama Y. Shigemasa K. Ohama K. GnRH agonist inhibits human telomerase reverse transcriptase mRNA expression in endometrial cancer cells. Intern J Molec Med 10: 593-597, 2002.
79. Imai A. Ohno T. Iida K. Fuseya T. Furui T. Tamaya T. Gonadotropin-releasing hormone receptor in gynecologic tumors. Frequent expression in adenocarcinoma histologic types. Cancer. 74(9):2555-61, 1994.
80. Schally A V. Nagy A. Chemotherapy targeted to cancers through tumoral hormone receptors. Trends Endocrin Metab 15: 300-310, 2004.
81. Aslam M. Fu L. Su M. Vijayamohanan K. Dravid V P. Novel one-step synthesis of amine-stabilized aqueous colloidal gold nanoparticles. J Mat Chem 14: 1795-1797, 2004.
82. Kakar S S. Winters S J. Zacharias W. Miller D M. Flynn S. Identification of distinct gene expression profiles associated with treatment of LbetaT2 cells with gonadotropin-releasing hormone agonist using microarray analysis. Gene 308: 67-77, 2003.
83. El-Naggar S M, Malik M T. Kakar S S. Small interfering RNA against PTTG: a novel therapy for ovarian cancer. Int J Oncol 31: 137-43, 2007.
84. Singal P K. and Iliskovic N. Doxorubicin-induced cardiomyopathy. N Engl J Med 339:900-905, 1998 85.
85. Menna P, Salvatorelli E and Minotti G (2008) Cardiotoxicity of antitumor drugs. Chem Res Toxicol. 21(5): 978-89, 2008.
86. Minotti G., Menna P., Salvatorelli E., Cairo G., Gianni L. Anthracyclines: Molecular Advances and Pharmacologic Developments in Antitumor Activity and Cardiotoxicity. Pharmacol Rev. 56(2): 185-229, 2004. Review
87. Gewirtz D A (1999) A critical evaluation of the mechanisms of action proposed for the antitumor effects of the anthracycline antibiotics adriamycin and daunorubicin. Biochem Pharmacol. 57(7):727-41, 1999. Review.
88. Wong E. and Giandomenico C M. Current status of platinum-based antitumor drugs. Chem Rev. 99 (9) 2451-2466, 1999.
89. Ranson M R. Carmichael, J., O'Byrne K., Stewart S., Smith D., Howell A. Treatment of advanced breast cancer with sterically stabilized liposomal doxorubicin: results of a multicenter phase II trial. J Clin Oncol. 15(10):3185-91, 1997.
90. Northfelt D W. Dezube, B J., Thommes J A, Miller B J., Fischl M A., Friedman-Kien A., Kaplan C D, Du Monte C, Mamelok R D, Henry, D H. Pegylated-liposomal doxorubicin versus doxorubicin, bleomycin, and vincristine in the treatment of AIDS-related Kaposi's sarcoma: results of a randomized phase III clinical trial. J Clin Oncol. 16(7):2445-51, 1998.
91. Gordon A N Fleagle J T., Guthrie D., Parkin D E, Gore M E, Lacave A J. Recurrent epithelial ovarian carcinoma: a randomized phase III study of pegylated liposomal doxorubicin versus topotecan. J Clin Oncol. 19(14):3312-22, 2001.
92. Muggia F et al, 2001; Liposomal encapsulated anthracyclines: new therapeutic horizons. Curr Oncol Rep. 3(2):156-62, 2001. Review.
93. O'Brien M E R., Wigler N., Inbar M., Rosso R., Grischke E., Santoro A., Catane R., Kieback D G., Tomczak P., Ackland S P., Orlandi F., Mellars L., Alland L., Tendler C. Reduced cardiotoxicity and comparable efficacy in a phase III trial of pegylated liposomal doxorubicin HCl (CAELYX/Doxil) versus conventional doxorubicin for first-line treatment of metastatic breast cancer. Ann Oncol. 15(3):440-9, 2004.
94. Fishburn C S. The pharmacology of PEGylation: balancing PD with P K to generate novel therapeutics. J Pharm Sci 97: 4167-4183, 2008.
95. Scott M D, Murad K L, Koumpouras F, Talbot M, Eaton J W. Chemical camouflage of antigenic determinants: stealth erythrocytes. Proc Natl Acad Sci USA 94: 7566-7571, 1997.
96. Owens D E 3rd, Eby J K, Jian Y, Peppas N A. Temperature-responsive polymer-gold nanocomposites as intelligent therapeutic systems. J Biomed Mater Res A 83: 692-695, 2007.
97. Kakar S S. (1998) Inhibition of growth and proliferation of EcRG293 cell line expressing high-affinity gonadotropin-releasing hormone (GnRH) receptor under the control of an inducible promoter by GnRH agonist (D-Lys6)GnRH and antagonist (Antide). Cancer Res 58: 4558-4560.
98. Kakar S S. Jennes L. Molecular cloning and characterization of the tumor transforming gene (TUTR1): a novel gene in human tumorigenesis. Cytogenet Cell Genet 84: 211-6, 1999.
99. Hamid T. Malik M T. Kakar S S. (2005) Ectopic expression of PTTG1/securin promotes tumorigenesis in human embryonic kidney cells. Molecular Cancer. 4(1):3, 1-13.
100. Nagarajah N S. Vigneswaran N. Zacharias W. (2004) Hypoxia-mediated apoptosis in oral carcinoma cells occurs via two independent pathways. Molec Cancer. 3: 38.
101. Jung C K. Yoo J. Jong Y J. Kim S, Chu I S. Yeom Y I, Choi J Y and IM DS (2006) Adenovirus-mediated transfer of siRNA against PTTG1 inhibits liver cancer cell growth in vitro and in vivo. Hepatology. 43(5):1042-1052.
102. Hamid T. Kakar S S. (2004) PTTG/securin activates expression of p53 and modulates its function. Molecular Cancer. 3:18, 1-13.
103. Fleiss, J L. Design and Analysis of Clinical Experiments. New York: John Wiley and Sons, 1986. 104. Cohen, J. Statistical Power Analysis for the Behavioral Sciences, 2nd Edition. New Jersey: Lawrence Erlbaum Associates Publishers, 1988.
104. Valentovic M A. Alejandro N. Carpenter B, Brown P I and Ramos K (2006) Streptozotocin (STZ) diabetes enhances benzo (alpha) pyrene induced renal injury in Sprague Dawley rats. Toxicol Lett. 164 (3): 214-20, 2006.
105. Trull A K. Facey S P. Rees G W. Wight D G. Noble-Jamieson G. Joughin C. Friend P J. Alexander G J. Serum alpha-glutathione S-transferase—a sensitive marker of hepatocellular damage associated with acute liver allograft rejection. Transplantation 58: 1345-51, 1994.
106. Speth, P. A., Q. G. van Hoesel, et al. Clinical pharmacokinetics of doxorubicin. Clin Pharmacokinetics 15: 15-31, 1988.
107. Allen, T. M., W. W. Cheng, et al. Pharmacokinetics and pharmacodynamics of lipidic nano-particles in cancer. Current Medicinal Chemistry-Anti-Cancer Agents 6(6): 513-23, 2006.
108. Fishburn, C. S. The pharmacology of PEGylation: balancing PD with PK to generate novel therapeutics. Journal of Pharm Sci 97: 4167-83, 2008.
109. Stewart S L, Querec T D, Ochman A R, Gruver B N, Babb J S, Wong T S, Koutroukides T, Pinola A D, Klein-Szanto A, Hamilton T C, Patriotis C. Characterization of a carcinogenesis rat model of ovarian peneoplasia. Cancer Res 64: 8177-8183, 2004.
110. The SAS System V9, Cary, N.C.: SAS Institute Inc., 2003.
111. Agresti A. Categorical data analysis, 2nd Ed. New York: Wiley and Sons, 2002.
112. Holm S. A Simple Sequentially Rejective Multiple Test Procedure. Scandinavian Journal of Statistics 6, 65-70, 1979.
113. Westfall P H, Tobias R D , Rom D, Wolfinger R D and Hochberg Y. Multiple Comparisons and Multiple Tests Using the SAS® System, Cary, N.C.: SAS Institute Inc., 1999.
114. Fenske et al. Ionophore-mediated uptake of ciprofloxacin and vincristine into large unilamellar vesicles exhibiting transmembrane ion gradients. Biochem Biophys. Acta. 1998, 1414: 188-2-4.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for treating a cancer in a subject, comprising:
identifying the subject in need of treatment for the cancer; and
administering to the subject an effective amount of a composition comprising a plurality of a chemotherapeutic agent and a plurality of a ligand for a luteinizing hormone-releasing hormone receptor, wherein each ligand and each chemotherapeutic agent are conjugated to a nanoparticle.

2. The method of claim 1, wherein the ligand for a luteinizing hormone-releasing hormone receptor comprises a luteinizing hormone-releasing hormone or an analog thereof.

3. The method of claim 2, wherein the ligand for a luteinizing hormone-releasing hormone receptor comprises a [D-Trp⁶] luteinizing hormone-releasing hormone.

4. The method of claim 1, wherein the plurality of the ligand for a luteinizing hormone-releasing hormone receptor comprises about 150 to about 250 molecules of the ligand.

5. The method of claim 4, wherein the plurality of the ligand for a luteinizing hormone-releasing hormone receptor comprises about 200 molecules of the ligand.

6. The method of claim 1, wherein the chemotherapeutic agent is selected from the group consisting of cisplatin, carboplatin, oxaliplatin, paclitaxel, topotecen, and doxorubicin.

7. The method of claim 6, wherein the chemotherapeutic agent comprises doxorubicin.

8. The method of claim 1, wherein the plurality of the chemotherapeutic agent comprises about 50 to about 100 molecules of the chemotherapeutic agent.

9. The method of claim 1, wherein the plurality of the chemotherapeutic agent comprises about 75 molecules of the chemotherapeutic agent.

10. The method of claim 1, wherein the nanoparticle is a gold nanoparticle or a liposome.

11. The method of claim 10, wherein the nanoparticle is a pure gold nanoparticle.

12. The method of claim 1, wherein a concentration of the chemotherapeutic agent administered to the subject is about 5 μg/kg to about 15 μg/kg.

13. The method of claim 12, wherein the concentration of the chemotherapeutic agent is about 9.6 μg/kg.

14. The method of claim 1, wherein the cancer expresses a luteinizing hormone-releasing hormone receptor.

15. The method of claim 14, wherein the cancer is selected from breast cancer, ovarian cancer, prostate cancer, liver cancer, endometrial cancer, pancreatic cancer, melanoma, testicular cancer, pituitary cancer, uterine cancer, Non-Hodgkin's lymphoma, and renal cell carcinoma.

16. The method of claim 1, wherein the composition is PEGylated.

17. The method of claim 1, wherein the plurality of the ligand for a luteinizing hormone-releasing hormone receptor comprises about 200 molecules of a [D-Trp⁶] luteinizing hormone-releasing hormone and the plurality of the chemotherapeutic agent comprises about 75 molecules of doxorubicin, and wherein the nanoparticle is a pure gold nanoparticle.

18. A method for detecting a cancer in a subject, comprising:
administering to the subject an effective amount of an imaging agent comprising a plurality of a ligand for a luteinizing hormone-releasing hormone receptor conjugated to a nanoparticle, wherein the ligand targets the imaging agent to the cancer in the subject; and
imaging the agent targeted to the cancer to thereby detect the cancer in the subject.

19. The method of claim 18, wherein the ligand for a luteinizing hormone-releasing hormone receptor comprises a luteinizing hormone-releasing hormone or an analog thereof.

20. The method of claim 19, wherein the ligand for a luteinizing hormone-releasing hormone receptor comprises a [D-Trp⁶] luteinizing hormone-releasing hormone.

21. The method of claim 18, wherein the nanoparticle is a pure gold nanoparticle.

22. The method of claim 18, wherein the cancer expresses a luteinizing hormone-releasing hormone receptor.

23. The method of claim 22, wherein the cancer is selected from breast cancer, ovarian cancer, prostate cancer, liver cancer, endometrial cancer, pancreatic cancer, melanoma, testicular cancer, pituitary cancer, uterine cancer, Non-Hodgkin's lymphoma, and renal cell carcinoma.

24. The method of claim 18, wherein the agent is imaged by magnetic resonance imaging.
